(19) United States
(12) Patent Application Publication
(10) Pub. No.: US 2026/0221238 A1
(43) Pub. Date: Jul. 30, 2026

(54) STRUCTURAL CHARACTERIZATION OF MATERIALS WITH KIKUCHI DIFFRACTION

(71) Applicant: Northwestern University, Evanston, IL (US)

(72) Inventors: Vinayak P. Dravid, Glenview, IL (US); Roberto Moreno Souza dos Reis, Chicago, IL (US); Alfred Nicholas Yan, Evanston, IL (US)

(21) Appl. No.: 19/429,366

(22) Filed: Dec. 22, 2025

Related U.S. Application Data
(60) Provisional application No. 63/737,375, filed on Dec. 20, 2024.

(51) Int. Cl. G16C 60/00; G06N 3/0464; G06N 3/088; G16C 20/70

(52) U.S. Cl. CPC G16C 60/00; G06N 3/0464; G06N 3/088; G16C 20/70

(57) ABSTRACT

A materials characterization system includes a memory configured to store training data that is based on dynamic simulations of crystal structures of materials. The training data includes one or more labels. The memory also stores a plurality of experimental diffraction patterns, where the experimental diffraction patterns are unlabeled. The system also includes a processor operatively coupled to the memory and configured to train a convolutional neural network (CNN) based on the stored training data and the experimental diffraction patterns, wherein the CNN is trained to provide a space group classification for an unknown diffraction pattern.

### CROSS-REFERENCE TO RELATED APPLICATION

The present application claims the priority benefit of U.S. Provisional Patent App. No. 63/737,375 filed on Dec. 20, 2024, the entire disclosure of which is incorporated herein by reference.

### BACKGROUND

Crystal structure and material phase information can be extracted from Kikuchi patterns obtained from electron diffraction. To extract information on crystal structure from a Kikuchi pattern, methods like Hough indexing, spherical indexing, and dynamic template matching can be utilized. These methods first generate a list of candidate crystal structures that the sample may contain, and then experimentally acquired diffraction patterns are matched to a best fitting crystal structure. There are other variations of the above methods, of which the vast majority involve the identification of candidate structures, diffraction simulations of candidate structures, and then matching experimental patterns to the simulated patterns.

### SUMMARY

An illustrative materials characterization system includes a memory configured to store simulated training data that is based on dynamic simulations of crystal structures of materials. The simulated training data includes one or more labels. The memory also stores a plurality of experimental diffraction patterns, where the experimental diffraction patterns are unlabeled. The system also includes a processor operatively coupled to the memory and configured to train a convolutional neural network (CNN) based on the stored simulation training data and the experimental diffraction patterns, wherein the CNN is trained to provide a space group classification for an unknown diffraction pattern.

In one embodiment, the system includes an electron backscattered diffraction (EBSD) system that generates the experimental diffraction patterns. In another embodiment, the system includes a transmission Kikuchi diffraction system that generates the experimental diffraction patterns. In one embodiment, the simulated training data originates from a materials information database. In another embodiment, the CNN is trained through unsupervised domain adaptation. In another embodiment, the unsupervised domain adaption comprises maximum classifier discrepancy. In another embodiment, the experimental diffraction patterns include a same set of classes that are present in the simulated training data.

In one embodiment, the processor is configured to adjust crystal symmetry labels of the stored simulation training data based at least in part on visual analysis of diffraction patterns within the stored simulation training data. In one embodiment, the visual analysis is a manual analysis. In another embodiment, the visual analysis is performed by the processor based on a crystal symmetry of a diffraction pattern after atoms associated with the diffraction pattern are set to have an identical atomic number. In another embodiment, the processor artificially resamples the experimental diffraction patterns to keep a class distribution balanced during training with unsupervised domain adaptation.

An illustrative method for characterizing materials includes storing, in a memory of a computing system, simulated training data that is based on dynamic simulations of crystal structures of materials, wherein the simulated training data includes one or more labels. The method also includes storing, in the memory, a plurality of experimental diffraction patterns, wherein the experimental diffraction patterns are unlabeled. The method also includes training, by a processor operatively coupled to the memory, a convolutional neural network (CNN) based on the stored simulation training data and the experimental diffraction patterns. The method further includes using the trained CNN to provide a space group classification for an unknown diffraction pattern.

In one embodiment, the method includes generating the experimental diffraction patterns with an electron backscattered diffraction (EBSD) system. In another embodiment, the method includes generating the experimental diffraction patterns with a transmission Kikuchi diffraction system. In one embodiment, the method includes obtaining the simulated training data from a materials information database. In another embodiment, the training comprises training the CNN through unsupervised domain adaptation. In one embodiment, the unsupervised domain adaption comprises maximum classifier discrepancy.

In one embodiment, the method includes adjusting, by the processor, crystal symmetry labels of the stored simulation training data based at least in part on visual analysis of diffraction patterns within the stored simulation training data. In another embodiment, the method includes performing, by the processor, the visual analysis based on a crystal symmetry of a diffraction pattern after atoms associated with the diffraction pattern are set to have an identical atomic number. The method can also include artificially resampling, by the processor, the experimental diffraction patterns to keep a class distribution balanced during training with unsupervised domain adaptation.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts a workflow for developing space group type classification models in accordance with an illustrative embodiment.

FIG. 2 depicts a confusion matrix that is calculated from pooling in all of the patterns from all 5 validation splits and their corresponding predictions by the model trained on their respective training datasets in accordance with an illustrative embodiment.

FIG. 3A is a plot in which misclassifications for different CsCl-type materials in the validation datasets are plotted as a function of AZ and Z in accordance with an illustrative embodiment.

FIG. 3B is a plot of misclassifications for NaCl structured materials in accordance with an illustrative embodiment.

FIG. 3C is a table that depicts accuracies broken down with different symmetries and number of elements in accordance with an illustrative embodiment.

FIG. 4 depicts performance of the Resnet18 model trained on a simulated test set of 18 materials found in the experimental data in accordance with an illustrative embodiment.

FIG. 5 depicts a table for model performance on 5-class classification in accordance with an illustrative embodiment.

FIG. 6 is a table that shows maximum classifier discrepancy performances with temperature-based data augmentation in accordance with an illustrative embodiment.

FIG. 7 depicts a comparison of accuracies for each material under the different data augmentation regimes, from the runs where simulations of the phases in the experimental data were included for training in accordance with an illustrative embodiment.

FIG. 8 is an example in which the accuracies achieved through MCD (trained with heavy data augmentation while including simulations of phases in experimental data) and from one of the models from Kaufmann et al. are compared in accordance with an illustrative embodiment.

FIG. 9A is a table that depicts discrepancies in ground truth between the online structural database (Materials Project) and reported Bravais lattice in accordance with an illustrative embodiment.

FIG. 9B is a table showing accuracies of space group type Fd-3m materials with a lattice parameter less than 6.30 A (or unit cell volume less than 250 A\hat{ }³) in accordance with an illustrative embodiment.

FIG. 9C is a table of maximal subgroups as well as their respective indices in accordance with an illustrative embodiment.

FIG. 9D is a table of accuracies per material for MCD trained 6 space group types in accordance with an illustrative embodiment.

FIG. 9E is a table depicting results of a CALM analysis for a handful of Al4Ni3 patterns in accordance with an illustrative embodiment.

FIG. 10 depicts an experimental pattern displayed with corresponding DynamicS (Bruker) simulations of Al3Ni2 and Al4Ni3, along with the normalized cross-correlation score in accordance with an illustrative embodiment.

FIG. 11 is a table that shows lattice solutions of Al4CoNi2 patterns, along with lattice parameters and angles, as well as deviations φ and δ from ideal lattice parameter ratios and ideal lattice angles in accordance with an illustrative embodiment.

FIG. 12 is an experimental pattern with corresponding simulated patterns of Ta at a similar orientation in accordance with an illustrative embodiment.

FIG. 13A depicts a few experimental Fe patterns showing overlapping grains in accordance with an illustrative embodiment.

FIG. 13B depicts a few Fe patterns with very poor pattern quality, which may be from excessive strain in accordance with an illustrative embodiment.

FIG. 13C depicts higher quality Fe patterns that were indexed by CALM as cubic in accordance with an illustrative embodiment.

FIG. 14 is a table that depicts CALM-derived lattice solutions for several Fe patterns in accordance with an illustrative embodiment.

FIG. 15A is a comparison with FIG. 4 in Kaufmann et al. in accordance with an illustrative embodiment.

FIG. 15B is a comparison with FIG. 5 in Kaufmann et al. in accordance with an illustrative embodiment.

FIG. 15C is a comparison with FIG. 6 in Kaufmann et al. in accordance with an illustrative embodiment.

FIG. 15D is a comparison with FIG. 7 in Kaufmann et al. in accordance with an illustrative embodiment.

FIG. 16A shows one example of an EBSD pattern from the Al4CoNi2 dataset in accordance with an illustrative embodiment.

FIG. 16B shows the band profile of the white line trace, where the global maxima are marked by vertical lines, but a prominent lower order reflection is visible in accordance with an illustrative embodiment.

FIG. 17 depicts components of the system in accordance with an illustrative embodiment.

FIG. 18 is a summary of the workflow for acquiring experimental patterns by the system in accordance with an illustrative embodiment.

FIG. 19 depicts a workflow for conducting high-throughput diffraction simulations in accordance with an illustrative embodiment.

FIG. 20 depicts workflows for neural network training (upper) and classification of diffraction patterns (lower) in accordance with an illustrative embodiment.

FIG. 21 depicts a table that displays the average performance of MCD in 8 different runs when classifying the space group of the chemically disordered equivalent structure in accordance with an illustrative embodiment.

FIG. 22A is a table that displays the accuracy breakdown of MCD for different materials in the test dataset after the model is trained with all available phases in accordance with an illustrative embodiment.

FIG. 22B is a table that displays the accuracy breakdown of MCD for different materials in the test dataset after the model is trained and the patterns from NiAl and Al have been removed from the experimental dataset in accordance with an illustrative embodiment.

FIG. 23 is a scatter plot that reveals the trend between accuracy and Image Quality (IQ) in accordance with an illustrative embodiment.

FIG. 24A is a table that displays the performance of Resnet18 on predicting the space group type of the chemically disordered equivalent structure when tested on simulated patterns of materials in the experimental dataset and fluorite-type structures are included in the simulated training data in accordance with an illustrative embodiment.

FIG. 24B is a table that displays the performance of Resnet18 on predicting the space group type of the chemically disordered equivalent structure when tested on simulated patterns of materials in the experimental dataset and without fluorite-type structures in training data in accordance with an illustrative embodiment.

FIG. 25 is a table that depicts the performance of MCD broken down by material on predicting space group types of the chemically disordered equivalent structure when NiAl, Al, FeNi3, and Ni3Al are removed from the experimental dataset in accordance with an illustrative embodiment.

FIG. 26A is a table that depicts the performance of MCD on predicting space group types of the chemically disordered equivalent structure when fluorite-type structures are removed from simulated training dataset and the experimental data includes all the phases available in accordance with an illustrative embodiment.

FIG. 26B is a table that depicts the performance of MCD on predicting space group types of the chemically disordered equivalent structure when fluorite-type structures are removed from simulated training dataset and the experimental data excludes patterns from NiAl and Al in accordance with an illustrative embodiment.

FIG. 27 depicts a confusion matrix that is calculated from pooling in all of the patterns from all 5 validation splits and their corresponding predictions by the model trained on their respective training dataset for the task of chemically disordered space group classification in accordance with an illustrative embodiment.

FIG. 28 is a table that depicts the performance of MCD on predicting space group types of the chemically disordered equivalent structure when Al4CoNi2 and Al are removed from the training data in accordance with an illustrative embodiment.

FIG. 29 is a table that depicts the performance of the MCD voting ensemble when tested on each phase after it was individually removed from the training data, after being tested on patterns from the same phases as the training data, as well as the number of models in the ensemble in accordance with an illustrative embodiment.

FIG. 30 is a table that depicts the accuracy of a voting ensemble trained using supervised learning with labeled experimental patterns after it was tested on each phase after it was individually removed from the training data in accordance with an illustrative embodiment.

FIG. 31A is a table that depicts the accuracies of ensemble models on light phases labeled with Im-3m after compositional disordering after including heavy phases labeled with Im-3m in the training data in accordance with an illustrative embodiment.

FIG. 31B is a table that depicts the accuracy of an ensemble model on heavy phases labeled with Im-3m after compositional disordering after only being trained on the light phases from Im-3m in accordance with an illustrative embodiment.

FIG. 32A is a table that depicts the accuracy of an ensemble model on the light phases labeled with Pm-3m after compositional disordering after being trained only on the heaviest phase from Pm-3m in accordance with an illustrative embodiment.

FIG. 32B is a table that depicts the accuracy of an ensemble model on the heaviest phases labeled with Pm-3m after compositional disordering after being trained only on the lightest phase from Pm-3m in accordance with an illustrative embodiment.

### DETAILED DESCRIPTION

A Kikuchi pattern refers to a diffraction pattern that forms when electrons scatter in a crystalline material. Methods such as Hough indexing, spherical indexing, and dynamic template matching can be utilized to extract information on crystal structure from a Kikuchi pattern. In Hough indexing, the Hough transformation is performed on the experimentally acquired patterns, from which Kikuchi line angles and positions are extracted. This is matched with the expected line angles and positions for each crystal structure in the candidate list, and the best fitting is assigned as the experimental pattern.

In dynamic template matching and dictionary indexing, Kikuchi pattern simulations are performed for the candidate crystal structures, and each experimental pattern is matched to a simulated pattern by calculating a similarity metric such as the normalized cross correlation. The crystal structure whose simulated patterns have the highest similarity with the experimental pattern is determined to be the crystal structure.

There are methods that can be used to directly identify the crystal symmetry from a Kikuchi pattern. For example, CALM is a software that requires the user to manually label the bands and zone axes (band intersections) in a Kikuchi pattern, and then calculates the Bravais lattice and lattice parameters. However, CALM does not give predictions for the space group, and the manual labeling process can be time-consuming.

Other researchers have attempted to train a neural network to predict the crystal symmetry of a sample using its diffraction pattern as input. However, past studies have only trained neural networks on experimental data, and not simulated data. This is not a practical solution, because it can be time-consuming and expensive to experimentally obtain diffraction patterns from a large number of materials to create a training dataset. Previous attempts to do this have not had high accuracies (30-50%).

High throughput crystal structure characterization has been attempted through scanning transmission electron microscopy (STEM). However, this method is not considered fast enough for quickly analyzing a large batch of materials. This is because of the low field-of-view in a STEM, such that only a small number of nanomaterials can be seen by the microscope at once, and characterizing millions of nanomaterials would involve a lot of moving around the sample. Other methods attempt to directly measure experimental properties of the resulting materials created from high-throughput synthesis. However, relevant material properties cannot always be determined through direct experimental measurement.

Described herein are fast and accurate methods for the crystal symmetry determination of novel material samples, which is necessary for the realization of high-throughput materials discovery workflows, such as with nanoparticle megalibrary platforms. One symmetry determination method involves collecting diffraction patterns through electron backscattered diffraction (EBSD) or transmission Kikuchi diffraction (TKD). Then, as an alternative to traditional symmetry determination methods, neural networks are used to quickly classify the crystal symmetry from such diffraction patterns. To demonstrate the feasibility of this solution, the inventors trained neural networks to predict the space-group type of background corrected EBSD patterns. The focus was on structures in the cubic point group m-3m, for which experimental data is available in the literature. The networks are first trained and tested on a dataset of high-throughput dynamical simulations in order to investigate the theoretical feasibility of space-group type determination using neural networks. Next, Maximum Classifier Discrepancy, an unsupervised deep learning domain adaptation method, is utilized to train neural networks to make predictions for experimental EBSD patterns. The developed models perform significantly better than the previous models in the literature, and suggest that neural networks are capable of making reasonable predictions of crystal symmetry from an EBSD pattern.

The advent of high-throughput synthesis techniques has recently unlocked the potential for combinatorial screening of millions of distinct materials for properties of interest. In particular, advances in scanning probe block copolymer nanolithography (SPBCL) enable the synthesis of millions of distinct nanostructures onto a substrate that is a few centimeters in length. The nanostructures can have a spatial gradient in both composition and size, which can be tuned by controlling the pen speed and ink composition during patterning. However, the ability to synthesize millions of distinct structures necessitates the development of high-throughput characterization techniques that can determine the corresponding sizes, shapes, and crystal symmetries, for which electron microscopy is considered to be an especially promising avenue.

To characterize the nanoparticles, methods for high throughput crystal symmetry determination are necessary. Deep learning methods can be used for this, as neural networks are able to take one or more diffraction patterns as an input and then output a prediction of the crystal symmetry within fractions of a second. Such an approach has been applied in electron microscopy, with a particular focus on 2D diffraction patterns like those from selected area electron diffraction (SAED) or convergent beam electron diffraction (CBED). In a recent study, Chen et al. utilized multiview opinion fusion machine learning to determine the crystal system from diffraction patterns taken from multiple zone axes, obtained from a scanning transmission electron microscope (STEM). Other studies on high-throughput symmetry determination use X-ray diffraction (XRD) patterns as an input. While some success has been found for the identification of crystal systems and space-group types, there have been difficulties at obtaining high classification accuracies when evaluated on large datasets with diverse ranges of crystal structures, especially when determining detailed descriptors like the space-group type.

Electron Backscattered Diffraction (EBSD) is an SEM surface mapping modality which may be an alternative method for high-throughput symmetry determination. One basic interpretation of EBSD is electrons backscattering off a sample while undergoing Bragg diffraction before colliding with a flat detector. Heavy multiple scattering occurs in EBSD, causing the electron signal to appear in the form of Kikuchi bands. As opposed to CBED or SAED, many zone axes are visible in the resulting pattern, which are represented by the intersections of Kikuchi bands. The standard application of EBSD is orientation mapping, which can be done at a very high volume using Hough-based indexing. This method extracts the bands in an EBSD pattern through the Hough transform, which allows a calculation of interplanar angles from the EBSD pattern, which is then matched to a list of candidate phases, e.g. through a procedure called triplet voting.

Beyond Hough indexing, many recent advances are based on dynamical EBSD simulations, which simulate multiple scattering using the Bloch wave approach and enable the fine details of an EBSD pattern to be reproduced. Dynamical simulations have been used to enhance orientation mapping through direct image correlation (DIC) methods, including dictionary indexing and spherical indexing, which involve matching an experimental pattern with EBSD simulations. Other DIC-based applications of EBSD, which often utilize pattern matching, include determination of chirality, polarity, and tetragonality. As with other electron diffraction modalities, studies have also utilized deep learning to aid EBSD analysis, ranging from applications like phase and orientation mapping.

A drawback of these traditional methods is dependence on prior knowledge of the sample phases, which may not be available for phases of novel materials. In these scenarios, there are a few existing options to extract information about the crystal symmetry or lattice without prior knowledge. These approaches return possible Bravais lattices and lattice parameters from analyzing the plane traces, zone axes, and bandwidths in a single EBSD pattern, which enables first principles predictions of the lattice metric without any prior knowledge of the phase.

However, for automated analysis on a large volume of patterns from different and possibly unknown phases, traditional techniques are not suitable, since at least a part of the bands still need to be assigned manually. Furthermore, traditional techniques do not give any space-group type predictions because of the complexity of Kikuchi-band profiles, and there is no reliable method of space group type determination for EBSD in the literature. Some attention was given to this area in recent studies, in which space group type determination is attempted through manual inspection. However, the reliable derivation of band profiles from a pattern was not demonstrated, and the manual inspection method was not proven. Since then, while there have been attempts to reconstruct atomic structures with EBSD, space group type determination via EBSD from first crystallographic principles has not received heavy attention even if it may be theoretically possible.

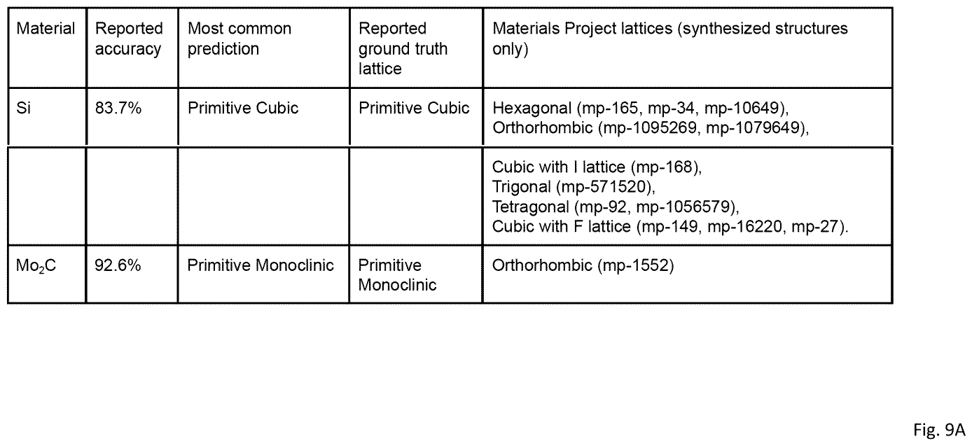

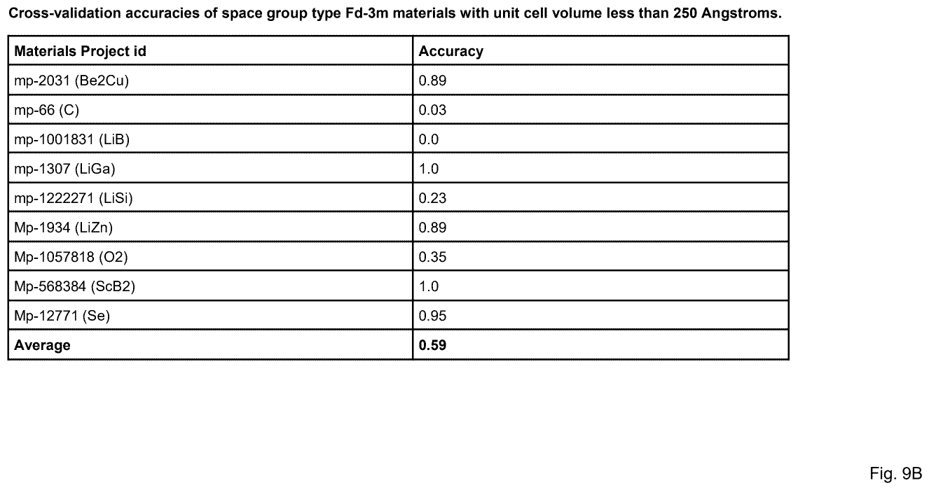

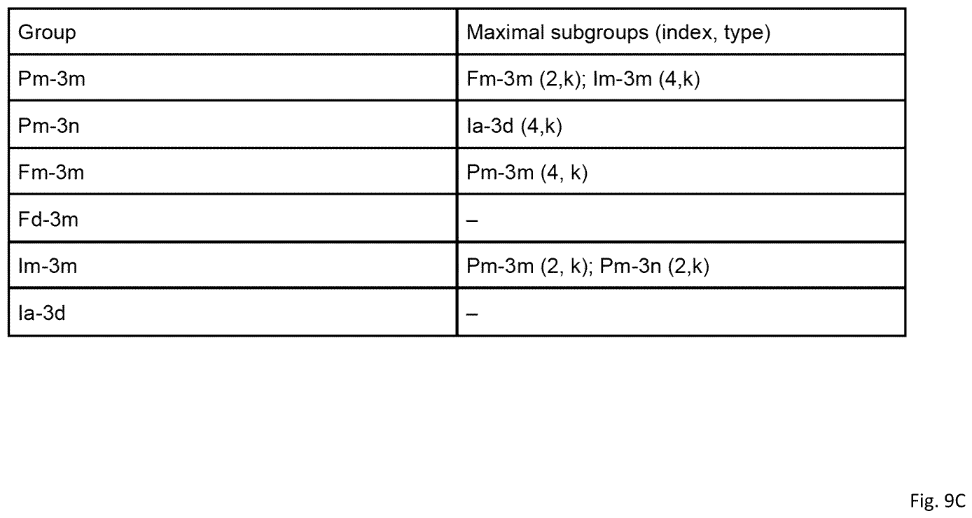

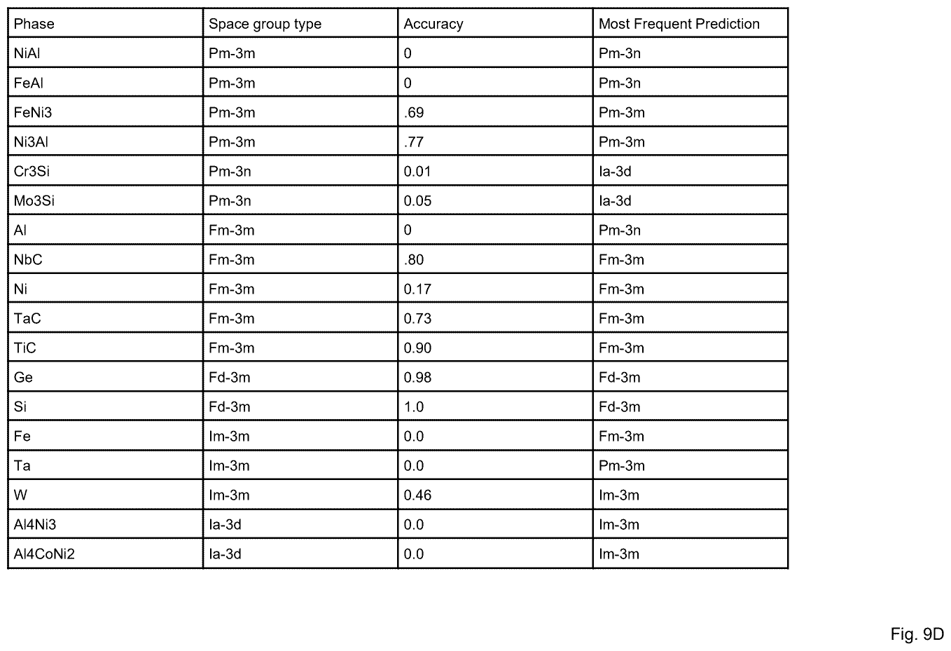

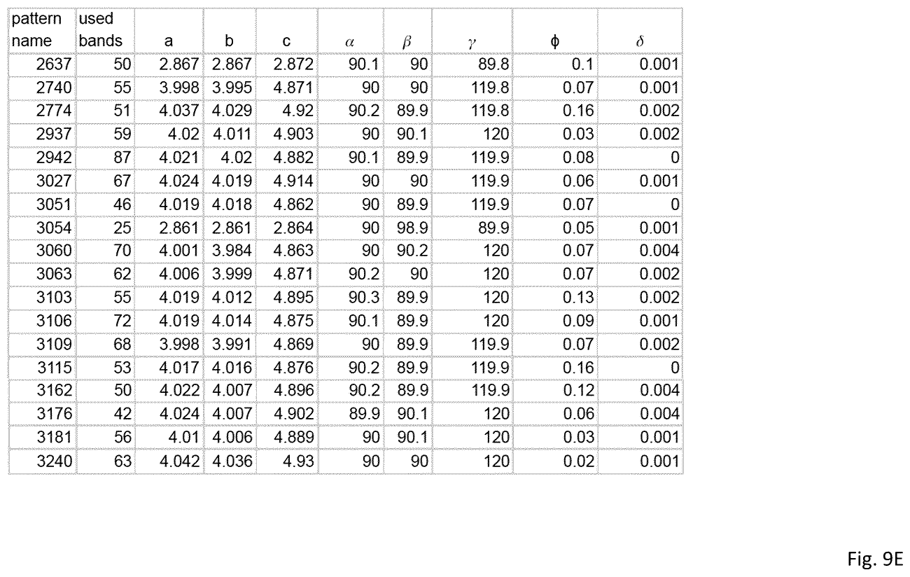

While the automation abilities of deep neural networks may alleviate the previous limitations, there has not been great success at training models to classify EBSD patterns based on symmetry. For example, Kaufmann et al. attempted to train convolutional neural networks to determine the space-group type of an EBSD pattern. Their training data included experimental EBSD patterns collected from 18 different material samples. However, they were not able to achieve reliably high performance when their models were tested on materials that are not in the training dataset. They have also attempted to train neural network models to predict the crystal system and Bravais lattice from EBSD patterns, though their models similarly displayed limited abilities at generalizing to novel materials (see FIG. 9A).

In the aforementioned studies, neural network misclassifications likely resulted from the low diversity of materials represented in the training dataset, such that a training dataset with a higher variety of chemical compositions, structure types, and lattice parameters could have improved performance. Usually, to improve material diversity for a training dataset of different crystal structures, high-throughput simulations are often used. For example, in Chen et al., STEM diffraction patterns are calculated for 3500 different materials for deep learning. However, for EBSD, while dynamical diffraction theory enables high quality simulations, they are time-consuming, and simulations for large datasets of different phases have rarely been performed in the literature. The closest demonstration of high-throughput simulation resulted from a study where simulations for around 350 different phases of mainly simple crystal structure types were performed. However, even if high-12 throughput simulations are performed, there are discrepancies between simulations and experiment, like noise, signal decay, and excess-deficiency effects, such that any neural network model trained to classify simulated EBSD patterns cannot presently make predictions for experimental patterns. Research on increasing the visual similarity between simulated and experimental images often utilizes image processing operations like contrast limited adaptive histogram equalization (CLAHE), or deep-learning based denoising. However, these still cannot yield simulated images that perfectly resemble experimental ones. Other research has successfully incorporated both simulated and experimental data using transfer learning and U-net image translation, but these methods are often used for orientation mapping where the material phase of the experimental pattern is known. Without such prior knowledge, as with combinatorial megalibraries with unknown phases, the efficacy of these methods is unproven.

The systems and methods described herein address the aforementioned issues by presenting a high-throughput dynamical EBSD simulation approach, paired with deep learning unsupervised domain adaptation methods to adapt models trained on simulations to experimental data, which enables the classification of experimental EBSD patterns from novel materials based on crystal symmetry. More specifically, this study develops convolutional neural network models for predicting the space group types given an EBSD pattern as input. The space group types 221 (Pm-3m), 223 (Pm-3n), 225 (Fm-3m), 227 (Fd-3m), 229 (Im-3m), and 230 (Ia-3d) from the cubic point group m-3m are included. Simulated training data is obtained through dynamical simulations of crystal structures from the Materials Project database. Next, models are trained to make predictions for experimental data through unsupervised domain adaptation. Unsupervised domain adaptation is a technique in deep learning which is used for training on a source dataset, for which labels are provided, and a target dataset, for which labels are absent. While the target dataset lacks labels, it contains the same set of classes as in the source dataset, such that a deep learning model could extract information from the labeled source dataset to train on the target dataset. Formally, the source and target dataset differ through covariate shift, which is bridged through domain adaptation training. In the proposed system, unsupervised domain adaptation is used to train on simulated patterns as the source dataset and experimental patterns as the target dataset in order to ultimately make predictions for the experimental patterns. As discussed in more detail below, the final model achieves accuracy scores on the experimental test set that are significantly greater than previous studies, suggesting that neural networks may have the potential to extract space group information from EBSD patterns even with experimental factors like noise and distortions.

The proposed methods and systems have been verified through experiments performed on test data. The experimental training and testing data used originate from Kaufmann et al., which are reported to contain EBSD patterns from 18 different materials from the 6 aforementioned space group types. A total of 144,465 patterns are in the testing dataset, and 3,000 patterns are contained in the combined training and validation datasets. The training, validation, and test data all contain patterns from the same 18 phases.

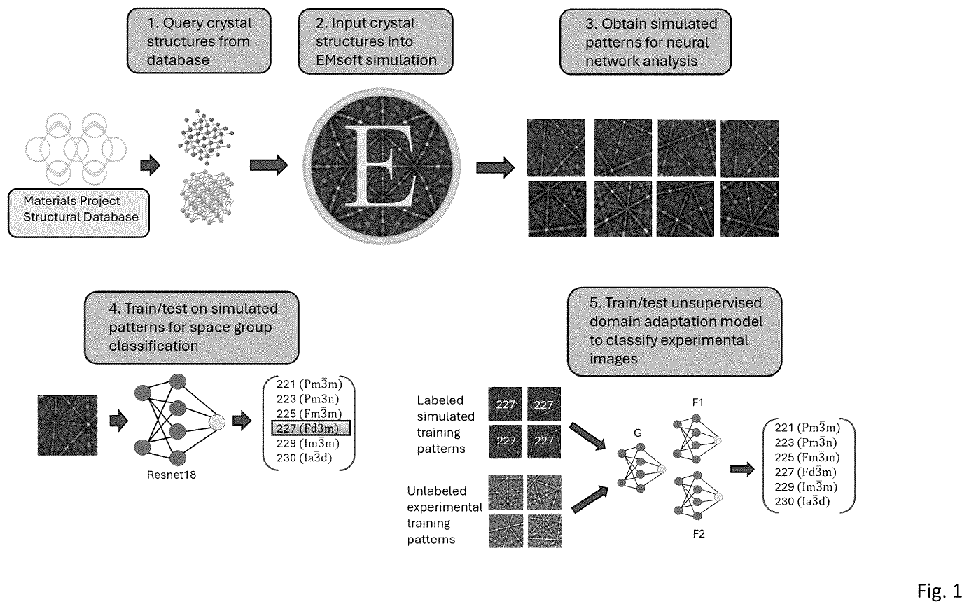

FIG. 1 depicts a workflow for developing space group type classification models in accordance with an illustrative embodiment. First, crystal structures were queried from the Materials Project database and their EBSD patterns are simulated using EMsoft. Alternatively, a different data source and/or simulation software may be used. The workflow incorporates some information about the lattice parameters of the phases represented in the experimental data. In particular, it was seen that in the experimental data, all the materials had a lattice parameter that was on the lower end of the lattice parameter frequency distribution for the space group type. Correspondingly, the model was trained on crystal structures with the lowest lattice parameters from each space group type in order to save computational resources. This was done by querying structures with a unit cell volume less than 250 Å³ for space group types Pm-3m, Pm-3n, Fm-3m, and Im-3m, and by querying structures with a unit cell volume less than 500 Å3 for space group type Fd-3m and 2000 Å³ for space group type Ia-3d, for which patterns with a unit cell volume less than 250 Å³ were too scarce. As used herein, a lattice parameter can refer to a fundamental measurement that defines the size and shape of a unit cell of the lattice, including edge lengths (e.g., a, b, c) and/or angles (e.g., α, β, γ) between the edge lengths.

To investigate whether there is space group information in EBSD patterns from a theoretical standpoint, a convolutional neural network (CNN) classification model with the Resnet 18 architecture was first trained and tested on simulated patterns only using 5-fold cross validation. Next, Maximum Classifier Discrepancy (MCD), which is an unsupervised domain adaptation technique, is used to train a model to predict the space group type for experimental datasets, using both the labeled simulated data and the unlabeled experimental data for training. The final model is evaluated on experimental data.

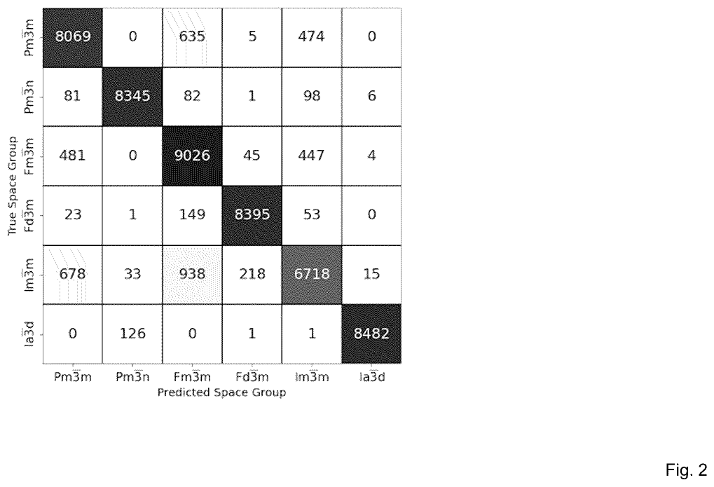

When the Resnet 18 model is evaluated on a simulated 5-fold cross-validation dataset, a balanced accuracy of 91% is obtained. FIG. 2 depicts a confusion matrix that is calculated from pooling in all of the patterns from all 5 validation splits and their corresponding predictions by the model trained on their respective training datasets in accordance with an illustrative embodiment. In FIG. 2, it can be seen that the model performs the worst on images from space group type Im-3m, and the best on images from space group type Fd-3m and Ia-3d.

The accuracy of 91% suggests the model has strong capabilities at distinguishing between low lattice parameter structures in each of the space group types. However, some limitations are observed when closely inspecting misclassifications. For example, the accuracy of structures in the space group type Fd-3m with a lattice parameter less than 6.30 Å is 68%, which is significantly lower than those with a higher lattice parameter (see FIG. 9B). This may be because of the scarcity of materials with a lattice parameter less than 6.30 Å (N=9) compared to those with greater (N=244). For each space group type Fd-3m material with a lattice parameter less than 6.30 Å, there may not be enough structures in its respective training dataset with a lattice parameter similar enough to it for the network to classify it correctly. The dependence on lattice parameters may originate from the dependence of EBSD band width on the d-spacing, which depends on the lattice parameter; materials with low lattice parameters may have unusually wide bands, such that the model may have trouble generalizing to them. Alternatively, some structures in the Fd-3m dataset have their atoms on a bcc lattice, like lithium boride (LiB), which makes them more likely to be misclassified.

Additionally, the unequal distribution of lattice parameters between space group types of the same Bravais lattice poses a risk of overfitting on lattice parameters instead of space group symmetry. For example, since the lattice parameters of most of the Fd-3m phases is greater than those of Fm-3m phases, the model can often trivially discriminate between the two symmetries using the lattice parameter. However, the model does well at differentiating between types Pm-3m, Pm-3n, Fm-3m, and Im-3m, which have the same lattice parameter cutoffs, suggesting that neural networks may still be able to distinguish between space group types or Bravais lattices of different phases even when they have similar lattice parameter distributions.

For these space group types, three are most frequently misclassified for each other (Pm-3m, Fm-3m, Im-3m). Interestingly, these three types are frequently k-type maximal subgroups of each other (see FIG. 9C), which suggests that phases in these space group types may fluidly transition between the three space groups whenever it loses or gains lattice centering. This may happen from changes in ordering. For example, in CsCl, if the Cs and Cl become disordered, then the phase transitions from Pm-3m to Im-3m.

Accordingly, different crystal structures in these space group types may become susceptible to misclassification if there is low differentiability in scattering factor between different elements in the structure, such that the exact ordering of different elements in the structure is hard to distinguish. Previous works on lattice determination of EBSD patterns have indeed found that pseudosymmetry frequently occurs when the differences in atomic scattering factors or atomic numbers of different elements in a crystal structure are too small. There was special emphasis on B2 type structures and B1 type structures, which are respectively from space group types Pm-3m and Fm-3m, and for which errors were predicted using empirical formulas.

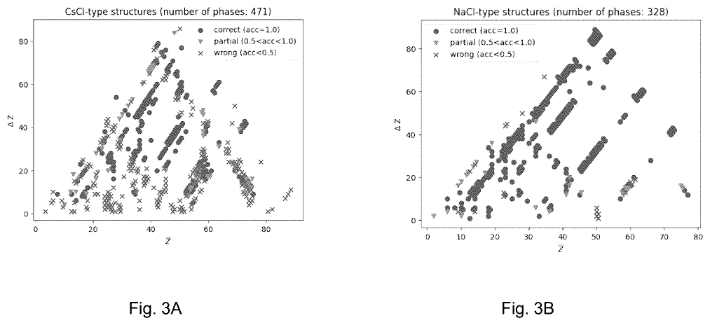

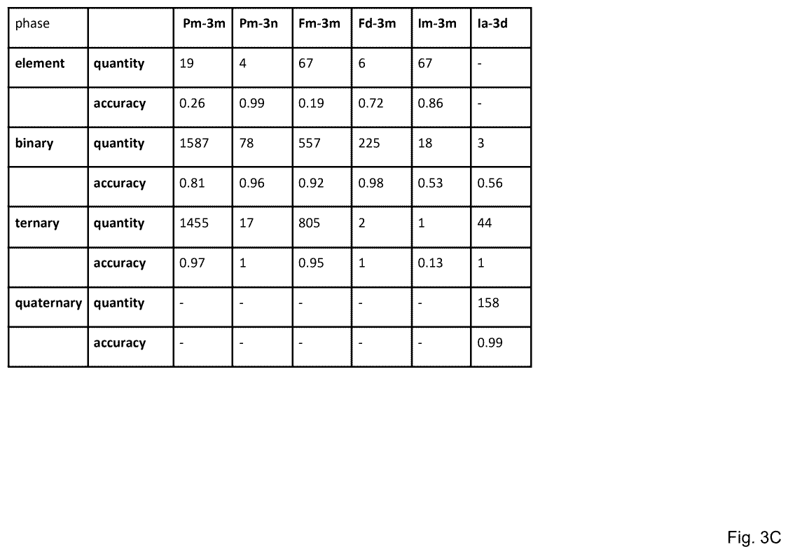

The proposed machine learning model may have similar trends when classifying B1 and B2 structures. For B2 structures, materials with lower differences in atomic number between the constituent elements are slightly more likely to be misclassified. FIG. 3A is a plot in which misclassifications for different CsCl-type materials in the validation datasets are plotted as a function of ΔZ (the difference in atomic number between the two constituent elements) and Z (the average atomic number between the two constituent elements) in accordance with an illustrative embodiment. In FIG. 3A, large clusters of misclassifications are seen at the bottom of the plot, where ΔZ is small. However, there can still be correct classifications when ΔZ is small, especially for B1 structures. FIG. 3B is a plot of misclassifications for sodium chloride (NaCl) structured materials in accordance with an illustrative embodiment.

Based on the results, the deep learning models seem less susceptible to misclassifying B1 structures. However, this may be because the space group type Fm-3m has one of the largest sampled number of crystal structures, which causes the model to frequently predict those types overall when faced with EBSD patterns that have an ambiguous lattice type. The high accuracy of predicting frequent structures may be a tradeoff for higher misclassification of other types of structures that are less common. This can be seen when inspecting the accuracies of phases in the validation sets, and dividing them by space group type and number of elements. FIG. 3C is a table that depicts accuracies broken down with different symmetries and number of elements in accordance with an illustrative embodiment.

The accuracy of simple cubic structures (with Ah structure type), for which around 19 phases exist, is low, which can be explained by their similarity in atomic positions and bond angles with B1 type structures. There may be several B1 type structures in the dataset that have low whose patterns appear similar to those from Ah type structures, ΔZ causing the model to match patterns from true Ah type structures to a B1 type structure.

Overall, the error profiles of deep learning and manual analysis software like CALM have similarities, as they are both caused by low differentiability in the atomic scattering factors of different elements, but the misclassifications of machine learning models may be biased towards predicting the space group types of the most frequent structure prototypes in the training dataset. Nevertheless, the correlation between misclassifications and maximal subgroup relations suggest that model error is systematic and can be anticipated.

Next, neural networks were trained using unsupervised domain adaptation to make predictions for experimental patterns. The Maximum Classifier Discrepancy (MCD) method was used to train on unlabeled experimental and labeled simulated images, with the simulations of the phases in the experimental data removed from the training data. The experimental training and test data originated from Kaufmann et al., and both datasets contain patterns from the same set of phases. Before model training, the experimental data was validated using manual crystallographic analysis through the CALM software.

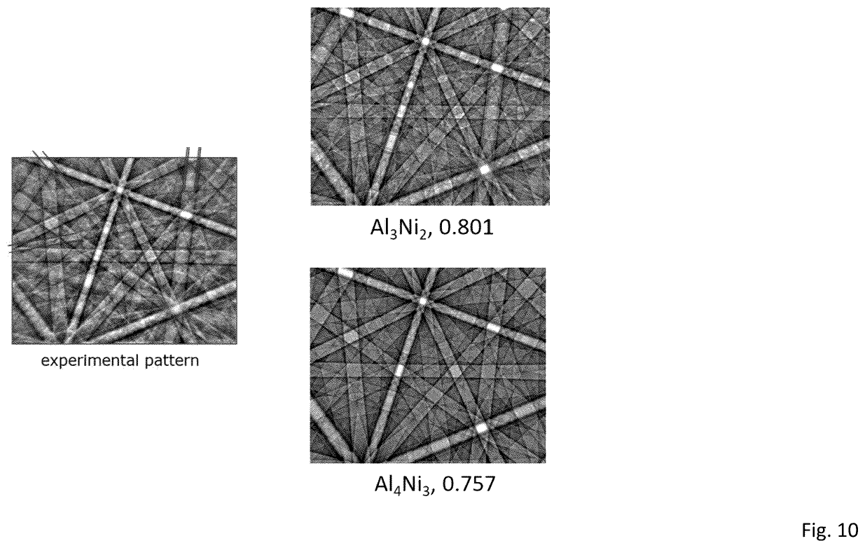

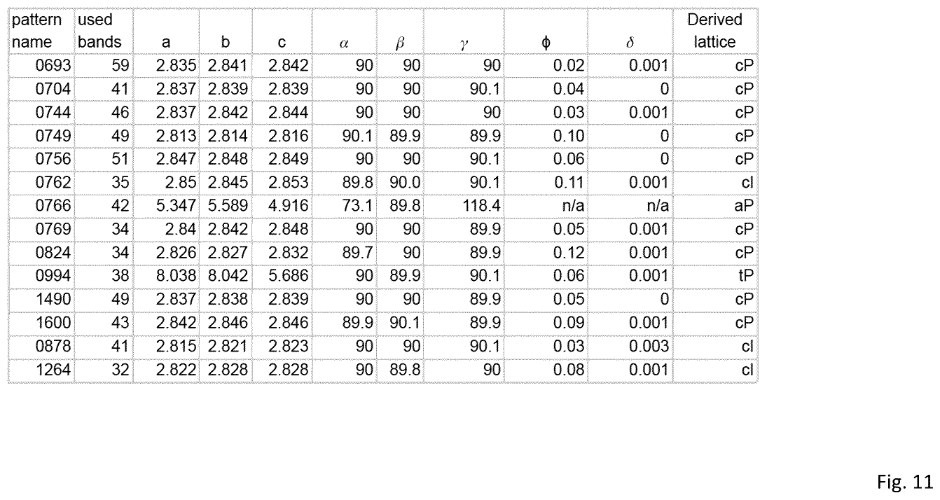

It was found that the patterns labeled as space group type Ia-3d did not reliably show the appropriate I lattices as expected. The two phases in the space group type Ia-3d, which were Al₄Ni₃ and Al₄CoNi₂, are superstructures marked by a CsCl-type structure with ordered vacancies at a range 4 times the lattice parameter of the usual CsCl-type structure. However, many patterns from Al₄Ni₃ indicated a hexagonal lattice (see FIG. 9E). The phase Al₃Ni₂ has trigonal symmetry, which has a hexagonal lattice, so it is possible that this phase was present in the sample. Furthermore, some Al₄Ni₃ patterns even contain visible bands that can be found in Al₃Ni₂ simulations and are absent in Al₄Ni₃ simulations, for which an example is in FIG. 10. Additionally, many patterns from the Al₄CoNi₂ patterns indicated a P lattice (see FIG. 11), with a lattice parameter close to that of (Co, Ni)Al, such that superstructure reflections from ordered vacancies were not found in the pattern and only sublattice was derived. An I lattice was derived for a few Al₄CoNi₂ patterns, but with the same lattice parameter of (Co, Ni)Al.

For these patterns for which an I lattice was derived, the ordered vacancies may have become disordered during sample preparation, resulting in a heavily defective (Co,Ni)Al structure which caused lower pattern quality. The reduction in pattern quality may have resulted in an incorrect derivation. Notably, the MCD model predicts the space group type Im-3m the most frequently for Al4CoNi2, which aligns with the I lattice derivation.

However, even if the sample were pristine, their resulting symmetry is expected to be very difficult to detect. This is because high thermal vibrations have been suggested to have a similar effect of pattern quality degradation as disordered defects, such that thermal diffuse scattering may mask the delicate symmetry resulting from ordered defects. Furthermore, in EBSD patterns, superstructure reflections appear as narrow low-intensity lower-order bands, and the reliable detection of such bands is noted to be challenging even with first-principles lattice analysis methods like CALM.

Overall, it is uncertain whether the samples from the space group type Ia-3d materials actually contain type Ia-3d symmetry. Even if they do, many materials in the space group type Ia-3d are characterized by superstructure reflections that are difficult to detect in EBSD, even by first-principle lattice analysis methods like CALM. Thus, it may be unreasonable to expect a neural network to classify such materials. Thus, the MCD model was retrained and tested only on the patterns from the space group types Pm-3m, Pm-3n, Fm-3m, Fd-3m, and Im-3m.

Furthermore, differences in symmetry due to chemical ordering are still difficult to detect in EBSD patterns. Thus, an alternative framework for symmetry determination is presented: instead of predicting the true space group, the models can be trained to predict the space group of the equivalent chemically disordered structure, obtained when all the atoms in the unit cell are changed to the same element. This is referred to as the “compositionally disordered space group”. Patterns from Al₄CoNi₂ are included for training and testing as well, and are treated as a bcc structure, which aligns with CALM analysis. To ensure high pattern quality, the training dataset was resampled. The Image Quality (IQ) of each pattern in the training and validation dataset was calculated, and the 100 highest quality images were sampled from each material from the combined training and validation datasets for MCD training.

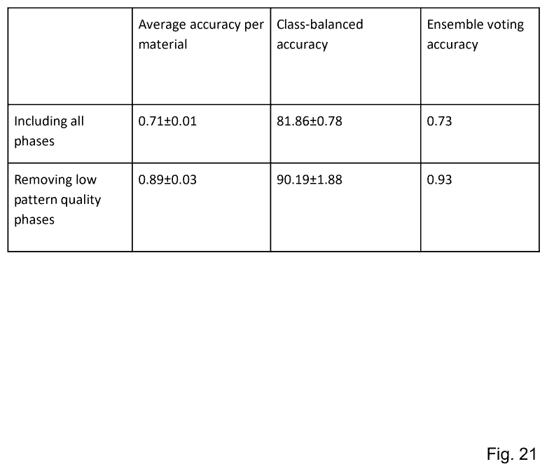

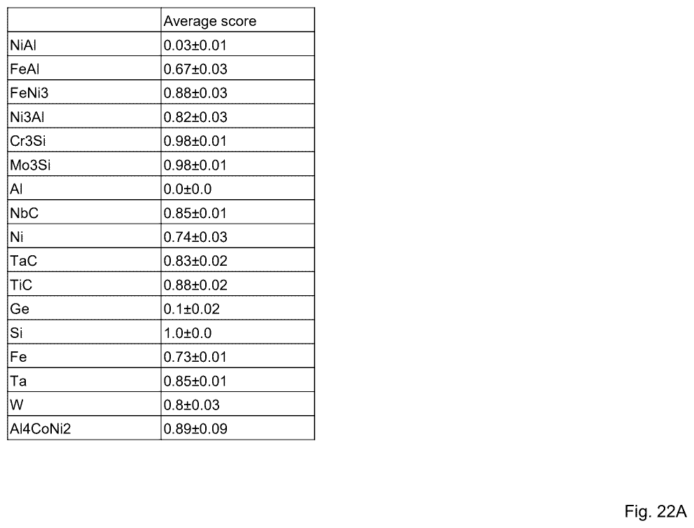

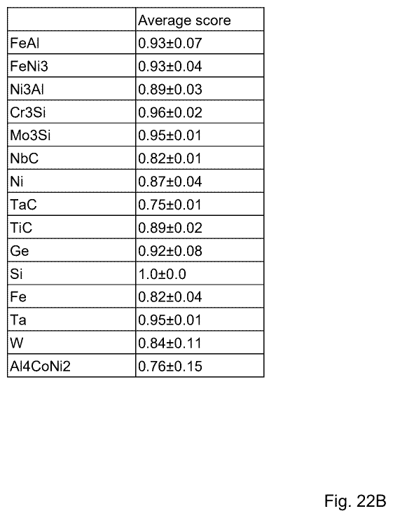

The MCD models are retrained accordingly for 8 different runs, and tested on the test dataset. A significant increase in accuracy is seen, as shown in FIG. 21. The accuracies from the individual runs are displayed in FIG. 22A and FIG. 22B. The accuracies are even higher with ensemble voting, when the predictions from all 8 models are considered for each test image and the most frequent prediction is chosen as a final overall prediction.

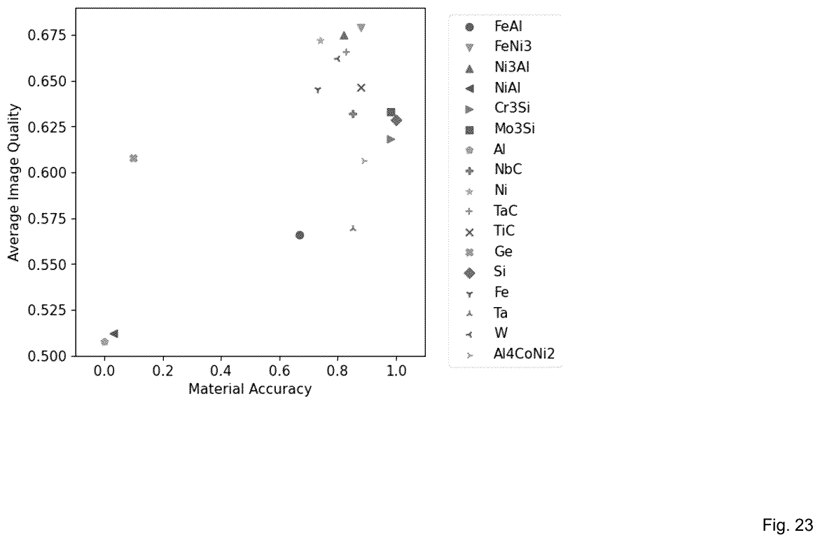

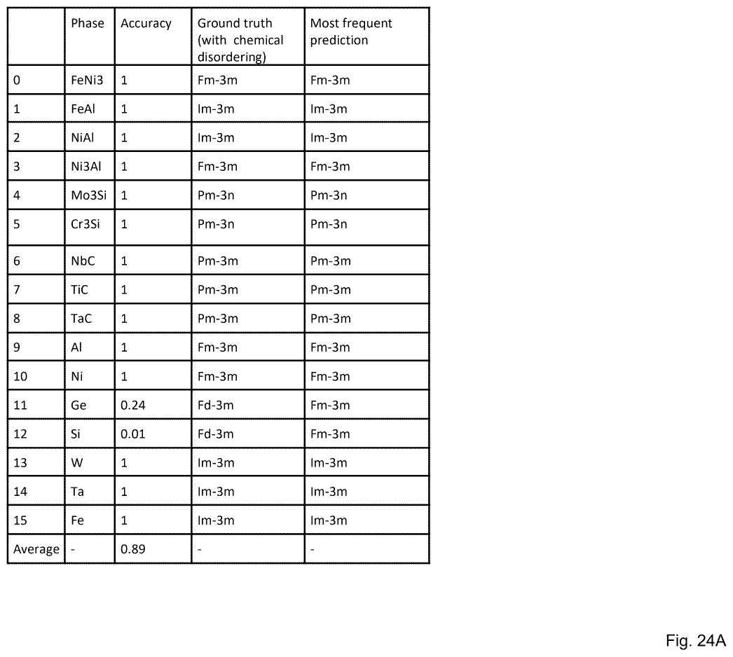

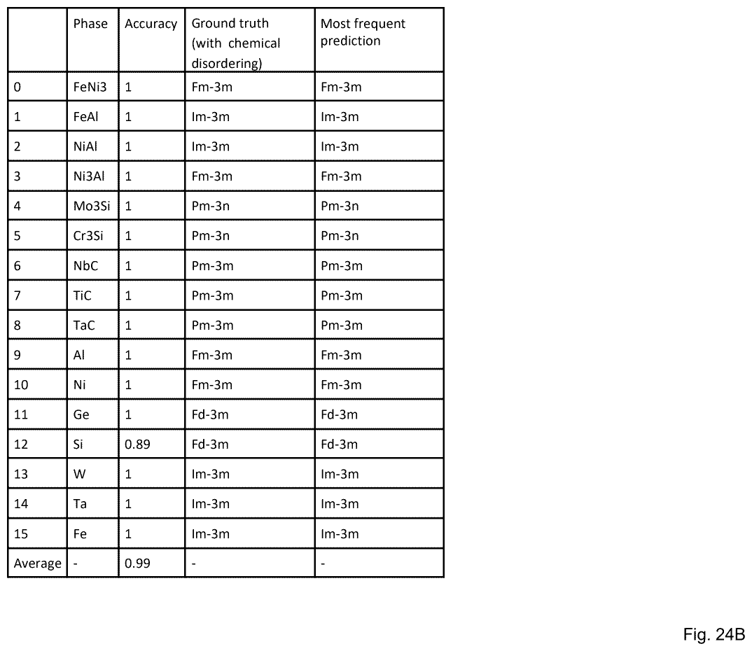

When examining misclassifications, the model only misclassified NiAl, Al, and Ge. Here, a strong correlation between Image Quality (IQ) and accuracy is observed-NiAl and Al have the lowest average IQs, which can be seen in FIG. 23. For NiAl and Al, it is likely that the low pattern quality is causing the misclassifications: the model correctly classifies Al₄CoNi₂, for which the pattern quality is higher and the reciprocal lattice is virtually identical to that of NiAl, and correctly classifies Ni, which is fcc like Al. Furthermore, when a Resnet18 model is trained and tested on simulated patterns, NiAl and Al are correctly classified, as shown in FIG. 24A. When the patterns from NiAl and Al are not included for training, the model performance increases significantly, and all the remaining phases have the majority of their patterns correctly classified.

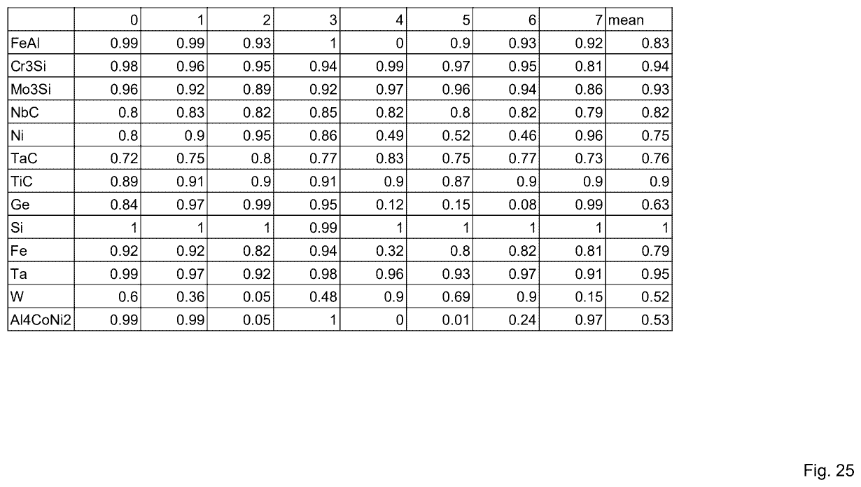

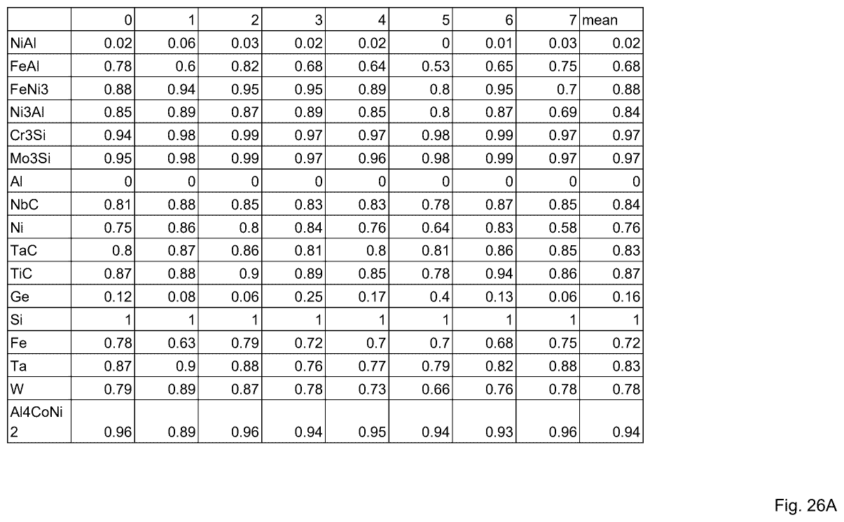

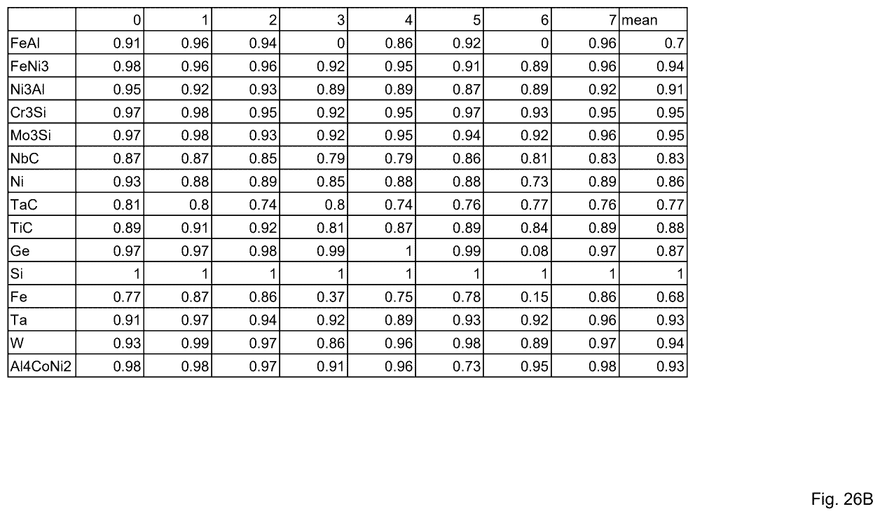

The initial misclassifications on Ge may be caused by the low-quality patterns from Ni and NiAl. The model frequently predicts NiAl and Al as Fd-3m, which is the same space group type as Ge. Incorrectly attributing the NiAl and Al patterns to Fd-3m likely caused the model to struggle with true Fd-3m materials-after retraining the model without NiAl and Al, the accuracy score for Ge increases to 0.87 (FIG. 22B), which suggests that filtering poor quality patterns may increase performance. Ge is also susceptible to misclassification because some fluorite-type structures in Fm-3m yield patterns that resemble those of Ge. In fact, after training and testing a CNN on simulated data, the space group type Fm-3m is incorrectly predicted for Ge and Si, but Fd-3m is correctly predicted when the fluorite-type structures are removed (FIG. 24B). This is because the model is trained to ignore chemical ordering, and the only difference between chemically disordered fluorite type structures and diamond structures is ordered vacancies in the 8c Wyckoff position of the fluorite structure. In fact, when patterns from FeNi3 and Ni3Al are removed from the training dataset, the model's performance slightly decreases again for Ge (FIG. 25), suggesting that it is difficult for it to differentiate between diamond and fluorite structured materials. However, when training MCD, removing the fluorite type structures from the training data doesn't improve performance on the experimental Ge patterns until the NiAl and Al patterns are also removed, as shown in FIG. 26A and FIG. 26B, suggesting that interference from the poor quality patterns from NiAl and Al is still significant.

Overall, after low-quality patterns are filtered out, the model shows success at differentiating between phases in the same Bravais lattice, such as between simple cubic substructures and A15-type structures in space group Pm-3n, as well as between fcc (A1 type) and diamond structures. If there is higher chemical ordering, which may change the true crystal symmetry, the neural network can still extract information on the reciprocal sublattice, yielding information on a structure prototype for which downstream electronic structure calculations or other relevant prior knowledge may be able to determine the exact ordering.

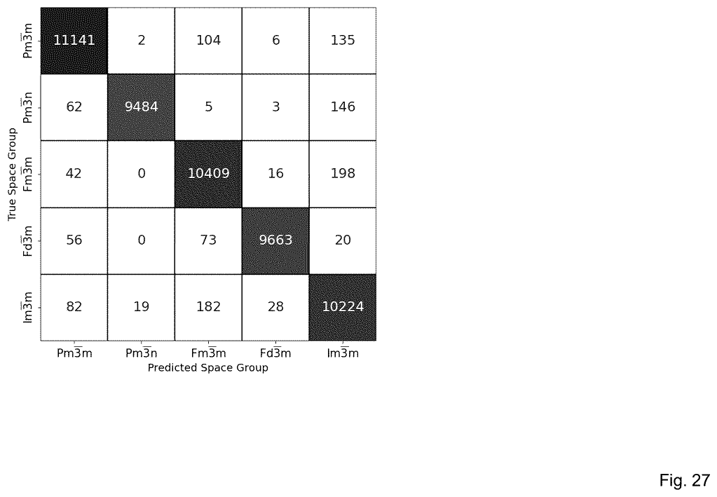

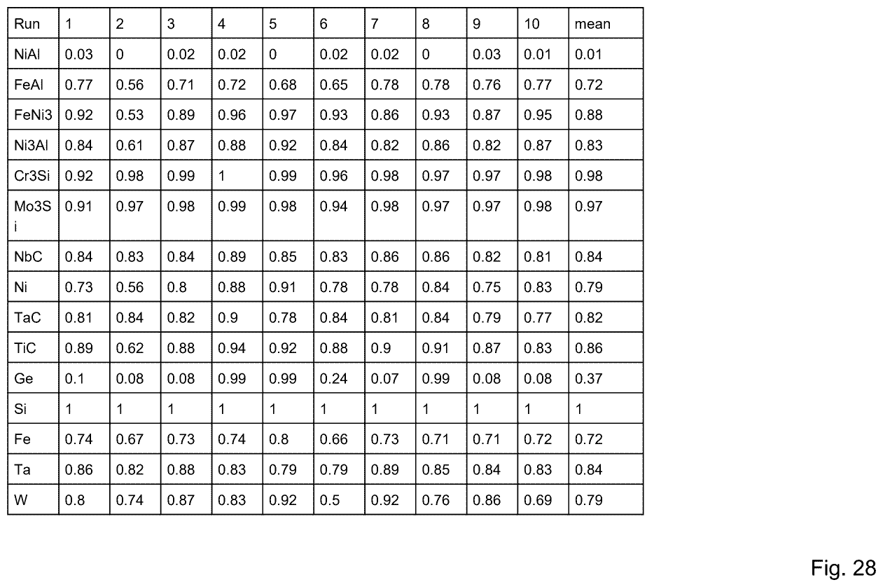

This approach of predicting the symmetry of the chemically disordered structure is expected to yield more consistent performance over predicting the true crystal symmetry. When trained and tested only on simulated patterns to predict the space group of the chemically disordered structure, the model yields a 98% cross-validation accuracy, shown in FIG. 27, demonstrating an increased consistency compared to the 91% accuracy when predicting the true space group.

Finally, neural networks are evaluated on experimental patterns from phases that are outside the training dataset. This is done using a method similar to leave-one-out cross-validation: for each phase in the experimental data, the representative experimental patterns were left out of the training data and 20 different models were trained using MCD on the remaining phases. Then, the models that yielded a high accuracy on the training patterns (i.e., achieved an accuracy above 50% on all the phases) were used to create a voting ensemble, where for each image the most frequent prediction was counted as a final prediction (with ties randomly decided). The ensemble was then evaluated on all the test patterns, including those from the phase excluded from training. During these runs, patterns from NiAl and Al were excluded due to low IQs, and with prior knowledge that fluorite structures can be confused with diamond structures, fluorite structures were removed from the simulated data in order to test the model's ability to differentiate diamond structures with fcc structures and the other structure prototypes.

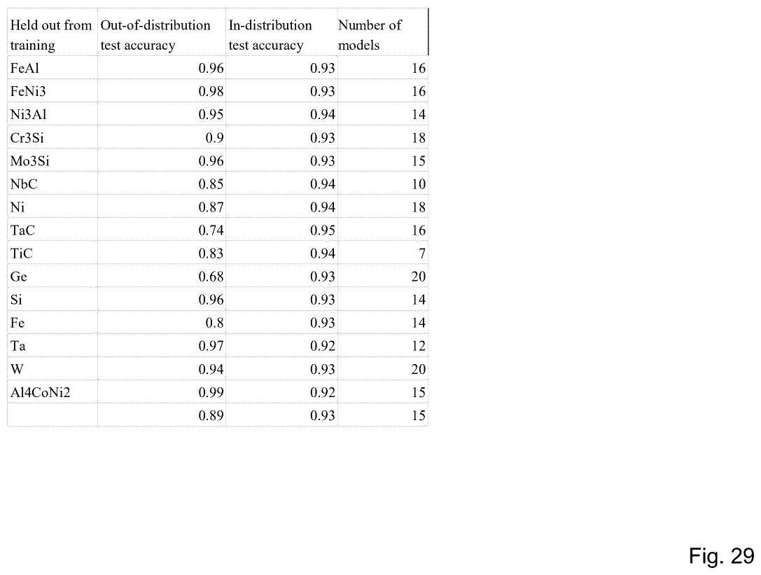

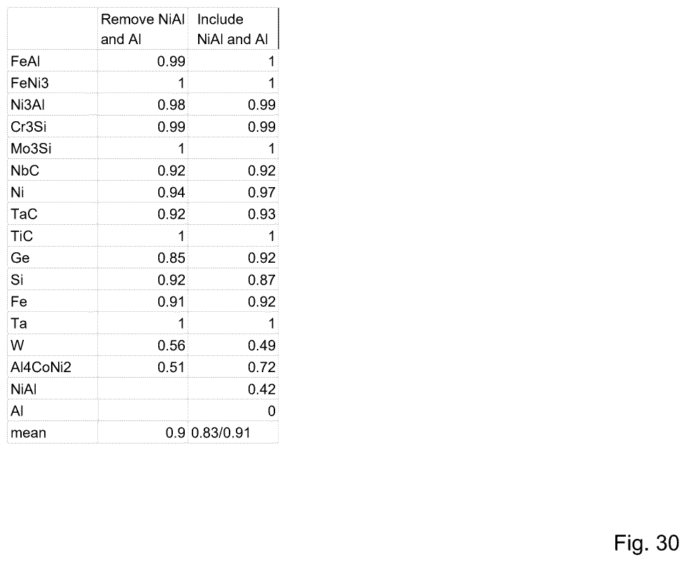

In FIG. 29, the accuracy of each phase in the test set is displayed, along with the corresponding accuracy of new patterns from the same phases in the experimental training data and the number of models selected for ensembling. The ensembles consistently perform well both on patterns from phases that were in the training data, as well as from novel phases that were outside the training data. For comparison, this procedure was also repeated by training an ensemble of Resnet50 CNN models directly on labeled experimental patterns without using simulated data. When training with labeled experimental patterns, high scores are also achieved, which are shown in FIG. 30, although there are slightly lower scores for one phase (W, Im-3m after disordering). The misclassification might originate from the resemblance between patterns from W and TaC (Pm-3m after disordering), as both phases contain elements with high atomic numbers and are expected to contain similar dynamical diffraction effects in their patterns.

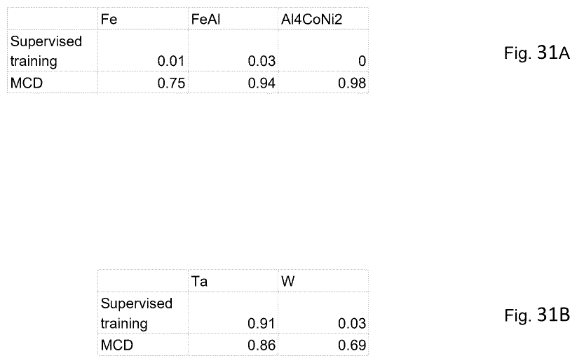

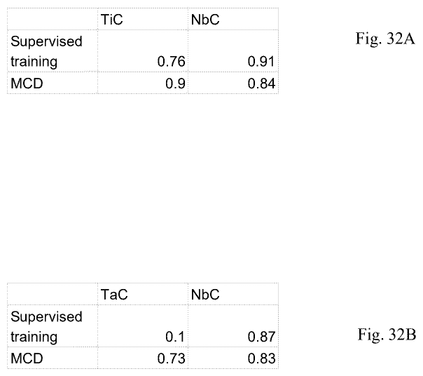

In this dataset, high accuracies may have been easier to obtain than in real-world experimental settings. This is because within each class, all the phases belonged to the same structure prototype after compositional disordering. Furthermore, within each class, there was often overlap in composition between the multi-elemental phases. For example, the Pm-3n phases all contained Si, and all the Fm-3m phases contained Ni. Nevertheless, even when there is a large difference in atomic numbers between the training and test set, high accuracies are still possible. To show this, it was first observed that there was a high variance of atomic numbers within Im-3m, with each phase either having high atomic numbers (Ta, W) or low atomic numbers (Fe, FeAl, Al₄CoNi₂). Accordingly, MCD and supervised classification models were evaluated on two dataset splits, one where the high Z phases in Im-3m (Ta, W) were used for testing and low Z phases (Fe, FeAl, Al₄CoNi₂) were used for training, and vice versa. As shown in FIGS. 31A and 31B, the MCD model ensemble performs significantly better than supervised learning on both these dataset splits, obtaining accuracies far above the baseline for each phase. Next, a similar procedure was done for the phases labeled Pm-3m, where the lightest (TiC) and heaviest phase (TaC) were each used as the only phase in Pm-3m for training and the model was tested on the other phases in the class. As shown in FIGS. 32A and 32B, MCD also achieves higher scores than supervised learning. The high scores for TaC are especially surprising because of the large difference in atomic number between Ta and C, such that the very high atomic scattering factor of Ta would typically pose a risk that the signal from C atoms is undetectable, even after the patterns are relabeled to space group 221. This suggests that the proposed deep learning workflow with pattern relabeling can bring improvements to the crystal symmetry information extractable from patterns. These results further suggest that training with unsupervised domain adaptation training using simulated data improves generalization to phases outside the experimental training dataset compared to supervised learning.

As discussed, EMsoft was used to generate dynamical simulations for the selected materials. First, .xtal files were created by reading data from each selected CIF file from the Materials Project database, which was automated using the pexpect python module. For the space group type Fd-3m, EMsoft requires the origin choice (1 or 2) to be specified. Thus, FINDSYM was used to create a new CIF file for each Fd-3m crystal structure, which had the origin choice explicitly specified in the file. Next, the xtal files were used to generate a master pattern for each material. Dynamical master pattern simulations were performed for a total of 5148 crystal structures. A small fraction of the materials in the space group type Ia-3d that did not finish after a 12 hour job were left out of the dataset. Finally, gnomonic projections were simulated using the master patterns in order to create images for training, validation, and testing. The sample-detector geometry used for the projections was set to be as close as possible to that of the experimental data. The sample-detector distance was set to 19100 microns, and the detector tilt was set to 13.7 degrees. The x and y coordinates of the pattern center and the pixel size were unknown. Thus, an arbitrary value for the pattern center from the literature was used, which was 171 microns above the detector center, and the pixel size was guessed to be 23 microns, which by inspection seemed to provide good agreement with experimental data. While close agreement between simulated and experimental parameters was desired, a perfect match was not required, as it was expected that the domain adaptation training would enable the neural networks to adjust to slight discrepancies.

The simulation input parameters were set to follow the experimental data as faithfully as possible; as the experimental accelerating voltage was 20 keV, each integer energy level from 10-20 keV was simulated. The minimum d-spacing was 0.05 nm. For the Bethe parameters, the strong beam cutoff used was 8.0, the weak beam cutoff was 50.0, and the complete cutoff was 100.0. Additionally, to reduce simulation time, the dimensions of the square master pattern were lowered to 601×601 pixels (npx=300). The simulation dwell time was set to 800 microseconds, and the beam current was set to 51 nA. For each phase, one simulation was completed where the Debye-Waller factor used was 0.005 nm² for all atoms in the unit cell, one where the Debye-Waller factor was randomly selected between 0.005 to 0.011 nm², and one where it was randomly selected from 0.005 to 0.017 nm².

Simulations were performed on a supercomputing cluster so that many batch jobs could be submitted in parallel. Furthermore, to decrease computation time, GPUs were used for the Monte Carlo simulations and 128 OpenMP threads were used for each master pattern calculation.

For both the space group classification task and the chemically disordered space group classification task, three simulated datasets were prepared from the patterns with a constant Debye-Waller factor of 0.005 nm²: a simulated cross-validation dataset, a simulated training dataset derived from the cross-validation dataset, as well as a simulated test dataset. To create the simulated test dataset, 100 gnomonic projections of random orientations were calculated from the master patterns of each of the phases in the experimental dataset. The master patterns of the rest of the phases were then used to generate a 5-fold cross-validation dataset: all the corresponding master patterns were randomly split into 5 folds, whose gnomonic projections made up 5 different train/test datasets for 5-fold cross-validation. This ensured that none of the phases in a validation dataset would also be present in the corresponding training dataset, enabling the evaluation of model performance on novel materials. Furthermore, due to the imbalance in the number of crystal structures simulated for each space group type, each type had a different number of gnomonic projections sampled per master pattern, such that the space group with the least phases would have 100 gnomonic projections per phase, and the number of projections per phase for the other space groups would result in a total number of projections that was as close as possible to the total number projections for the space group with the least phases. After creating the test and cross-23 validation dataset, the final training dataset for MCD training was created by combining the training and validation data from an arbitrary fold of the cross-validation dataset.

A training dataset from the master patterns that had varying Debye-Waller factors was created in a similar way to use during MCD training. Cross-validation and test datasets were not created from these master patterns.

First, neural networks with the Resnet18 architecture pre-trained on ImageNet were trained and tested on simulated images to predict the true space group as well as the compositionally disordered space group. Pattern simulations with uniform Debye-Waller factors of 0.005 nm² were used for training and testing. During training, the simulated images were resized to an image resolution of 229×229 pixels to be input into the model, which were randomly cropped into size 224×224 images during training. Data augmentation was also used by randomly flipping images up-down and left-right. Stochastic gradient descent was used for training, with a learning rate of 0.001 and with a momentum of 0.9. The learning rate decreased by 10% every 7 epochs. First, the model was trained on the cross-validation datasets for 10 epochs, after which the optimal number of epochs to train was determined from the epoch with the highest accuracy score. Then, a new model was trained on the respective combined training data and tested on the simulated test dataset. The optimal number of epochs was 10 for compositionally disordered space group type classification.

Next, Maximum Classifier Discrepancy (MCD) with a ResNet50 backbone pre-trained on ImageNet to predict the space group type of experimental EBSD patterns using unsupervised domain adaptation. This method involves adversarial training between two discriminators and a generator. One training step contains three distinct stages: in one stage, target images are input into the generator, and the generator output is input into both the discriminators. With the weights of the generator frozen, the weights of the discriminators are then updated to maximize the discrepancy between the outputs of the two discriminators, as well as classify the source images by minimizing cross-entropy loss. The discrepancy is a measure of the dissimilarity between the two output vectors, and is calculated as the L1 distance between the pair of vectors.

In the second stage, the discriminator weights are frozen and the generator is trained to minimize the discrepancy. This stage is repeated 4 times for each mini-batch (n=4), which was found to yield the highest performance in the original study. The final stage involves training both discriminator-generator pairs to classify the source images by minimizing cross-entropy loss.

In the proposed implementation, the generator can be a ResNet50 backbone, and the classifiers each consist of a single perceptron layer. Models were trained using the Adam optimizer with a learning rate of 0.0002 and batch size of 128, following the original implementation. For the initial runs on predicting the compositionally disordered space group where the model was trained and tested on patterns from all the phases available, the model was trained for 5 epochs. During the rest of the runs where phases were removed from the training data to be used for testing, training was done for 10 epochs. Additionally, no class balance loss term was used as done in the original MCD study, since the assumption of balanced classes in the experimental data did not hold.

During training to predict the compositionally disordered space group, the simulated dataset included both patterns with high Debye-Waller factors (0.005-0.011 nm2), as well as patterns with low Debye-Waller factors (0.005 nm2). The one exception is in the runs where Im-3m was separated into light and heavy phases and the heavy phases (W, Ta) were in the test set-in these runs, the high Debye-Waller factor simulations were not used due to high pattern quality of the experimental data. In the runs where different individual phases were held out for testing, simulated patterns from NiAl and Al were added to the simulated training data, since those phases were not in the experimental test data.

The experimental training dataset was created by combining the training and validation sets from Kaufmann et al. However, to ensure high pattern quality, the Image Quality (IQ) of each pattern was calculated, and the 100 highest quality images were sampled from each phase for training. In the separate runs where each phase was individually removed from the training set to be used for testing, when a phase was removed, 100 more images from the rest of the phases in the class were added to the training data to keep the classes sufficiently balanced to enable a high accuracy on the training data. These images were divided evenly among the remaining phases and were selected based on highest IQs. However, this was not done for the Im-3m class due to a large number of phases already in this class. In the separate runs where the class Pm-3m was separated into heavy and light phases, only one phase was available in the class Pm-3m for training. Thus, the patterns from this phase were oversampled by adding each pattern from the 100 highest IQ patterns to the dataset 3 times.

Finally, neural networks with the Resnet50 architecture pre-trained on ImageNet were trained on experimental patterns only to predict the compositionally disordered space group. This was done using a leave-one-out evaluation method as in MCD, as well as separating light and heavy phases in Pm-3m and Im-3m. The same images were included in the dataset as the MCD runs in order to enable direct comparison between the two methods. For each classification task, 20 models were trained for 10 epochs each and were all used to form a voting ensemble.

CALM was used to analyze the patterns in the experimental space group type Ia-3d dataset. Many lattice plane traces and zone axes are selected in each pattern, and then automatic bandwidth detection is applied to derive a lattice solution. However, because automated bandwidth selection could overlook local extrema in band profiles that represent lower-order interferences, lower order interferences were searched for afterwards to obtain another refined lattice solution which may be different from that obtained through automated bandwidth detection. An example of a band profile from an Al4CoNi2 pattern containing lower-order interferences is shown in FIG. 16.

Additional information regarding the experiments are included in FIGS. 9A-9E, which are referenced in the discussion above. FIG. 9A is a table that depicts discrepancies in ground truth between the online structural database (Materials Project) and reported Bravais lattice in accordance with an illustrative embodiment. FIG. 9B is a table showing accuracies of space group type Fd- 3m materials with a lattice parameter less than 6.30 A (or unit cell volume less than 250 A\hat{ }³) in accordance with an illustrative embodiment. FIG. 9C is a table of maximal subgroups as well as their respective indices in accordance with an illustrative embodiment. Only the 6 space group types in the experiments are included, and it is noted that these space group types are all klassengleichen subgroups of each other. FIG. 9D is a table of accuracies per material for MCD trained 6 space group types in accordance with an illustrative embodiment. FIG. 9E is a table depicting results of a CALM analysis for a handful of Al4Ni3 patterns in accordance with an illustrative embodiment. The lattice parameters a, b, and c are given as well as the angles α, β, and γ. Φ describes the deviation in lattice parameter ratio from the ideal lattice, and δ describes the deviation in lattice angles from the ideal values.

FIG. 10 depicts an experimental pattern displayed with corresponding DynamicS (Bruker) simulations of Al3Ni2 and Al4Ni33, along with the normalized cross-correlation score in accordance with an illustrative embodiment. The Al3Ni2 has a higher score, suggesting greater similarity with the experimental pattern. Some visible features in the experimental pattern that can be seen exclusively in the Al3Ni2 simulation are highlighted with lines that extend outward from the image.

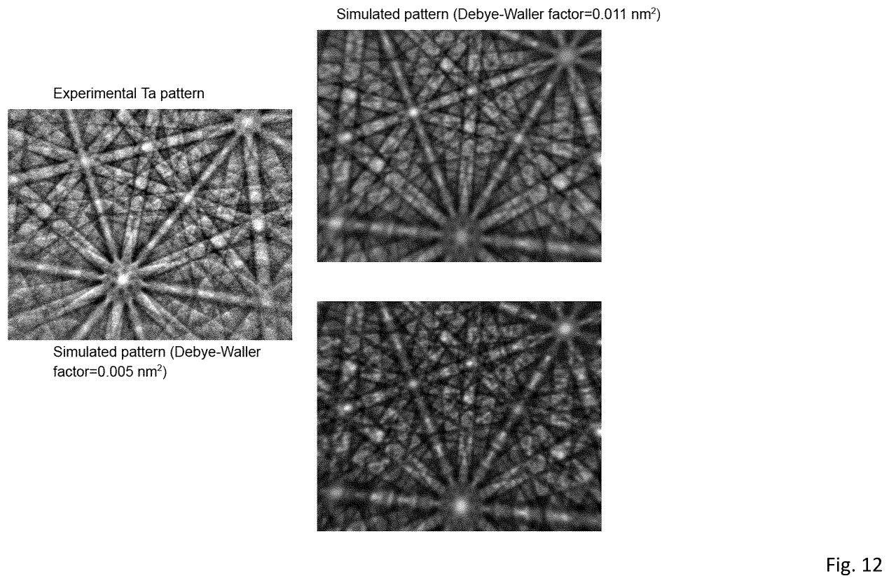

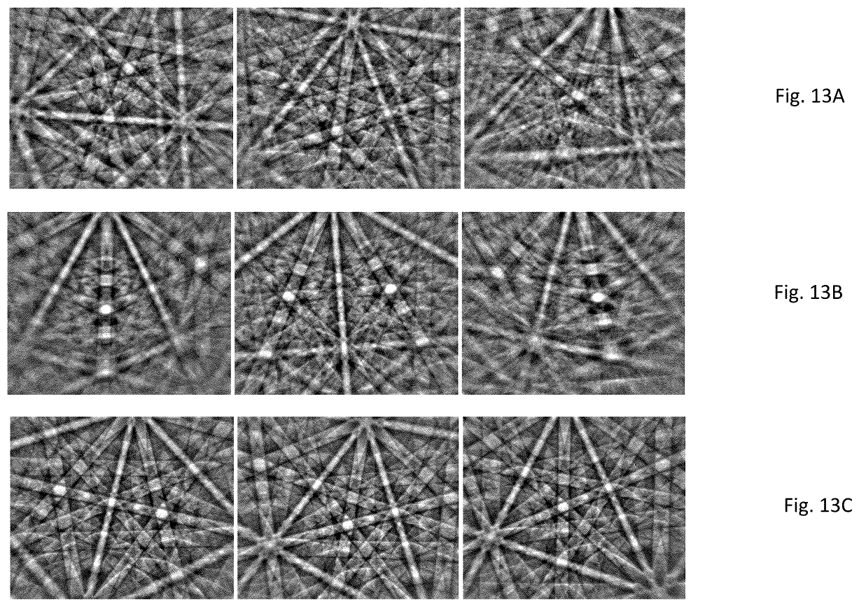

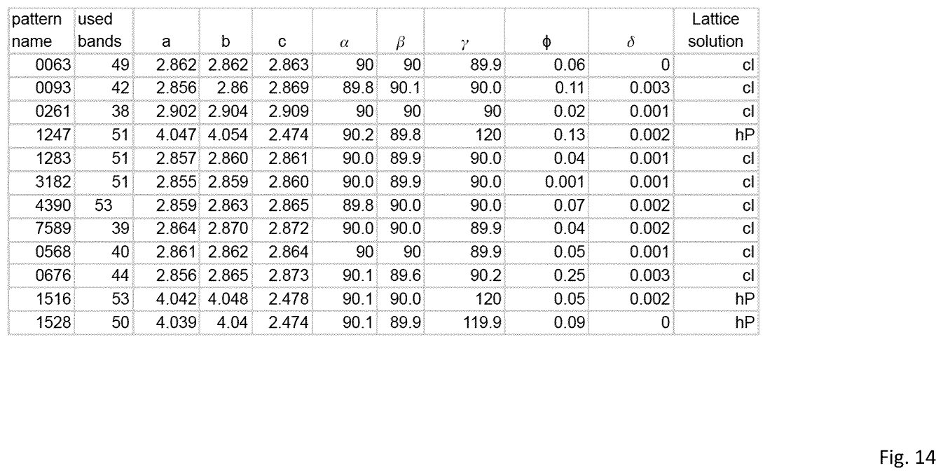

FIG. 11 is a table that shows lattice solutions of Al4CoNi2 patterns, along with lattice parameters and angles, as well as deviations φ and δ from ideal lattice parameter ratios and ideal lattice angles in accordance with an illustrative embodiment. FIG. 12 is an experimental pattern with corresponding simulated patterns of Ta at a similar orientation in accordance with an illustrative embodiment. FIG. 13A depicts a few experimental Fe patterns showing overlapping grains in accordance with an illustrative embodiment. FIG. 13B depicts a few Fe patterns with very poor pattern quality, which may be from excessive strain in accordance with an illustrative embodiment. FIG. 13C depicts higher quality Fe patterns that were indexed by CALM as cubic in accordance with an illustrative embodiment. FIG. 14 is a table that depicts CALM-derived lattice solutions for several Fe patterns in accordance with an illustrative embodiment.

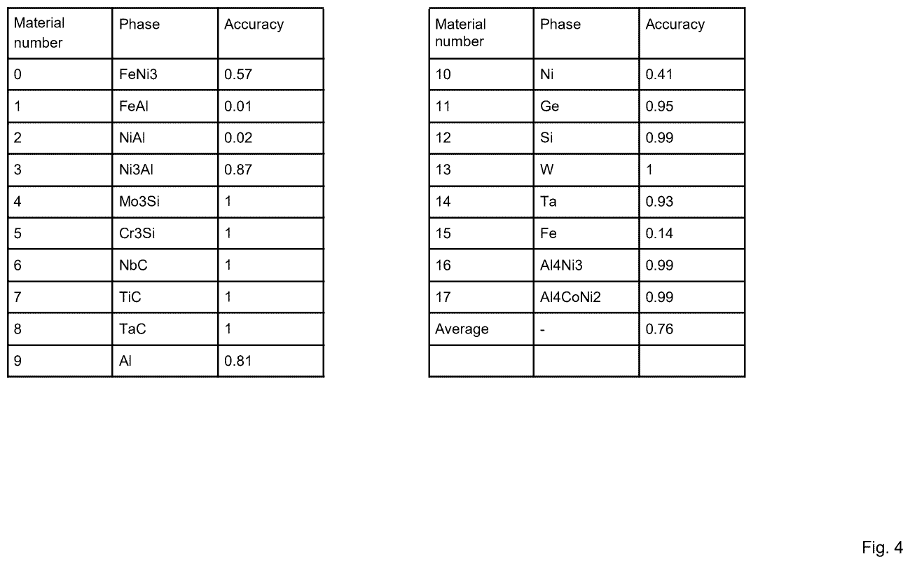

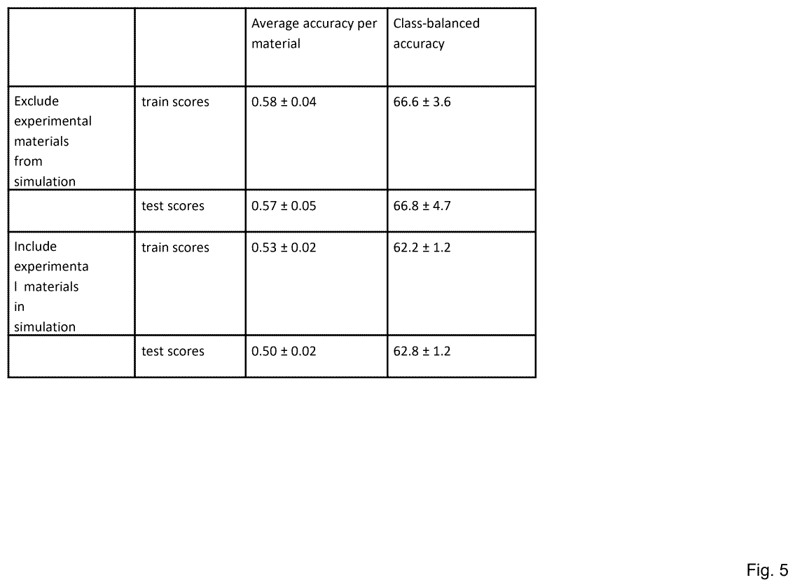

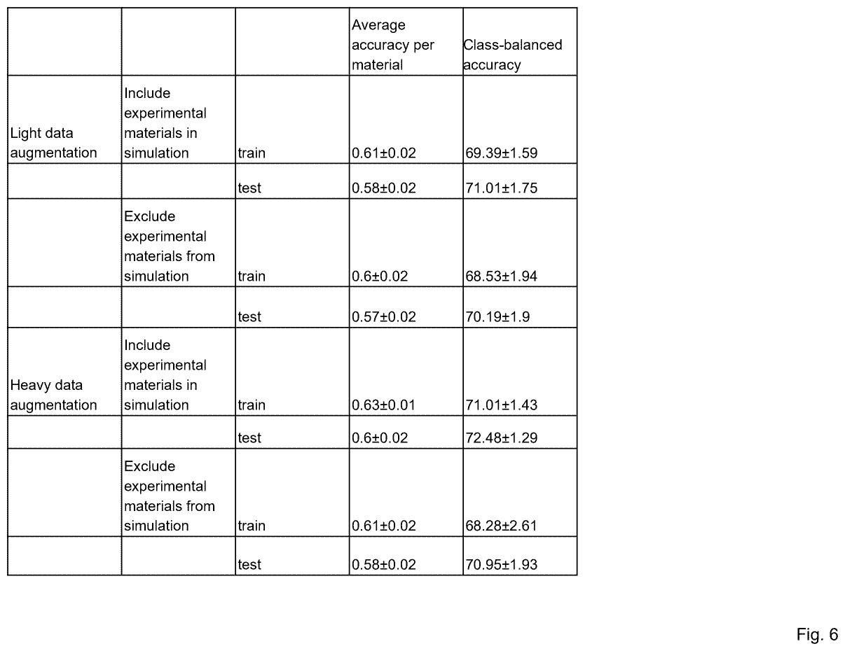

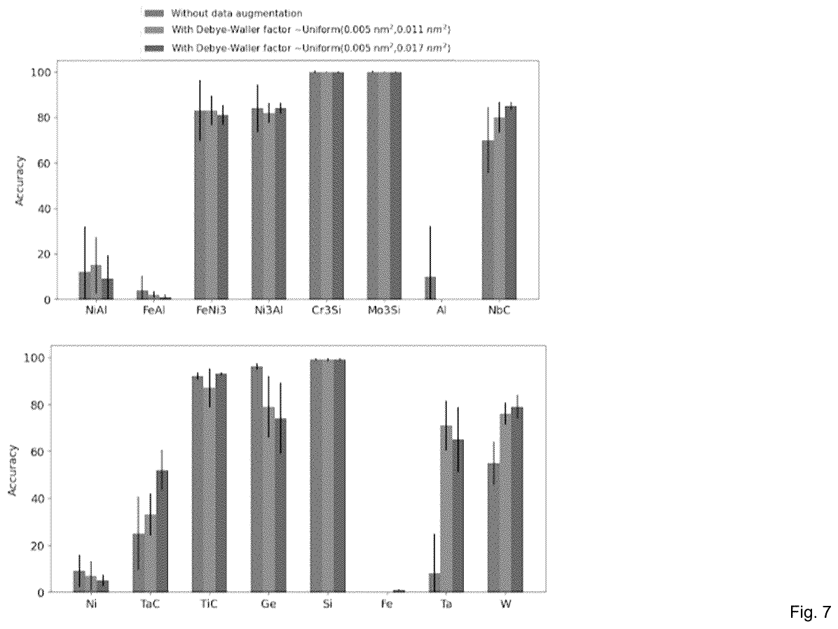

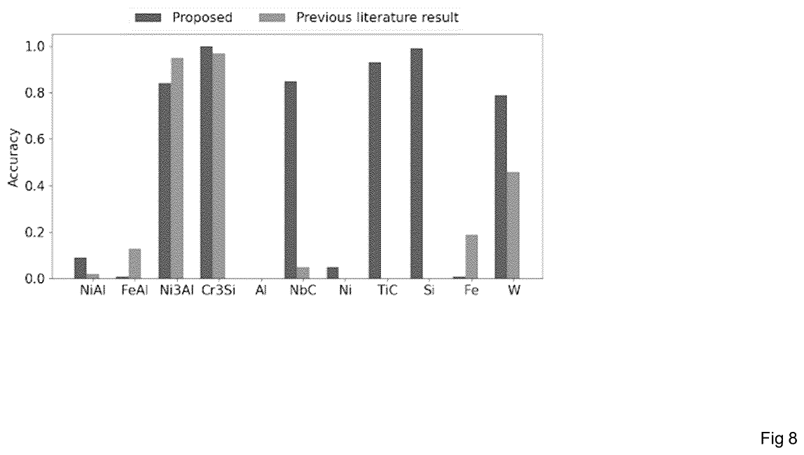

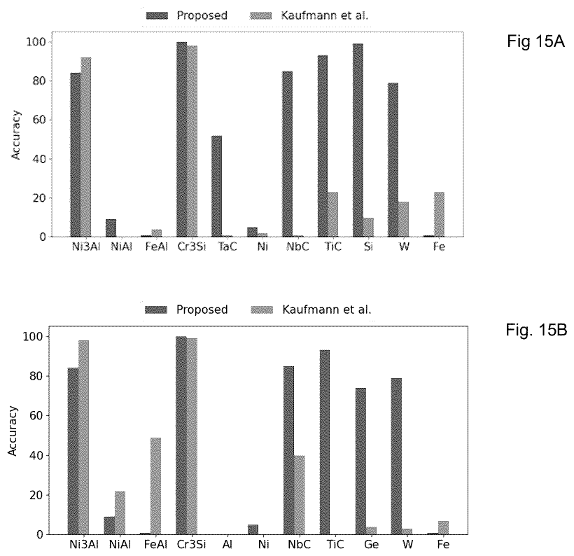

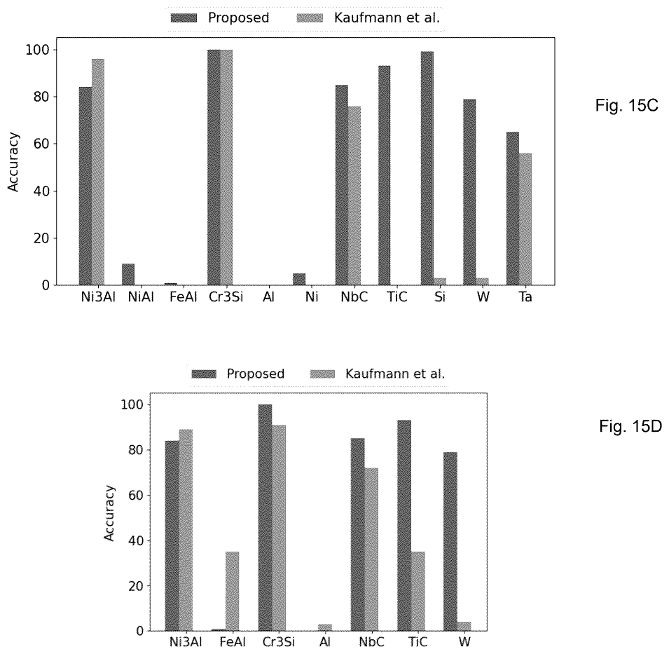

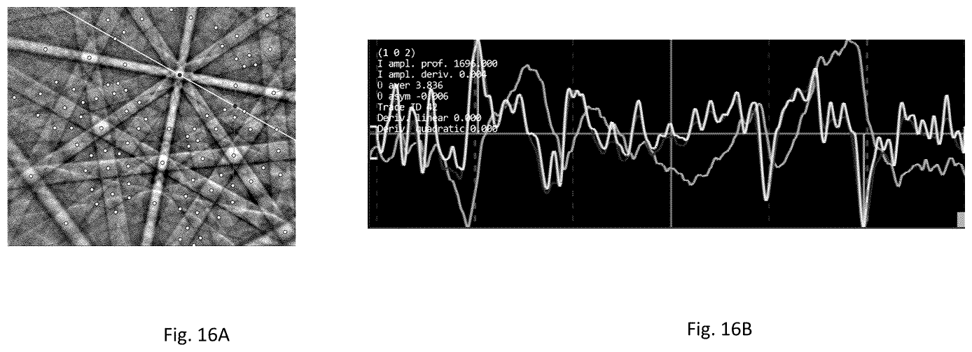

FIG. 15 shows plots comparing the accuracies of the Maximum Classifier Discrepancy model and the models developed by Kaufmann et al. FIG. 15A is a comparison with FIG. 4 in Kaufmann et al. in accordance with an illustrative embodiment. FIG. 15B is a comparison with FIG. 5 in Kaufmann et al. in accordance with an illustrative embodiment. FIG. 15C is a comparison with FIG. 6 in Kaufmann et al. in accordance with an illustrative embodiment. FIG. 15D is a comparison with FIG. 7 in Kaufmann et al. in accordance with an illustrative embodiment. FIG. 16A shows one example of an EBSD pattern from the Al4CoNi2 dataset in accordance with an illustrative embodiment. Selected zone axes are marked by the white dots. FIG. 16B shows the band profile of the white line trace, where the global maxima are marked by vertical lines, but a prominent lower order reflection is visible in accordance with an illustrative embodiment.

Described in more detail below is the system for implementing the methods described above. The system implements a process that enables structural characterization of a material sample, and the determination of crystal symmetry or other related crystal structure properties. As discussed above, this process is anticipated to be useful for the characterization of samples for which there is little prior information known about the specific crystal structures present, and when the crystal structure for a very large number of distinct, previously unknown materials in the sample need to be determined. The system can utilize a materials characterization device, like an electron microscope, to acquire a collection of Kikuchi diffraction patterns from a sample. In the most common implementation, the patterns are collected by transmission Kikuchi diffraction using a scanning electron microscope. Afterwards, a computer is used to generate a large collection of crystal structures that may be representative of the material sample, as well as the corresponding labels for the crystal structures, which are values of a descriptor of interest for which it is desired to analyze from the experimental diffraction patterns. This can include crystal structure descriptors such as lattice parameters, lattice angles, Bravais lattice, or space group. Next, the computer is used to perform Kikuchi diffraction simulations for the collection of crystal structures. A neural network is trained on a combination of the experimental patterns, the simulated patterns, as well as the labels for the simulated patterns, in order to predict the descriptor of interest for each experimental pattern. The neural network is trained using an unsupervised domain adaptation method, such as the Maximum Classifier Discrepancy algorithm. After neural network training, the neural network outputs a prediction for the descriptor of interest for the experimental patterns, enabling elucidation of the crystal structure of the sample. In one embodiment, after the initial prediction, an algorithm based on crystallographic first principles then is used to determine alternative predictions in case the first prediction is incorrect.

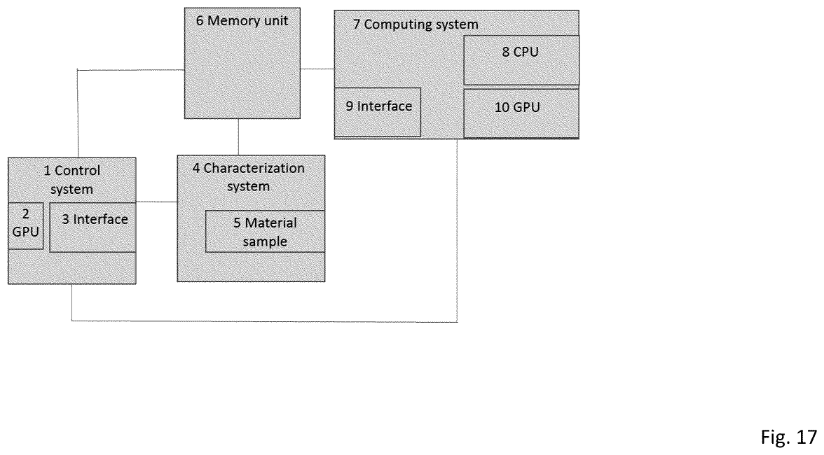

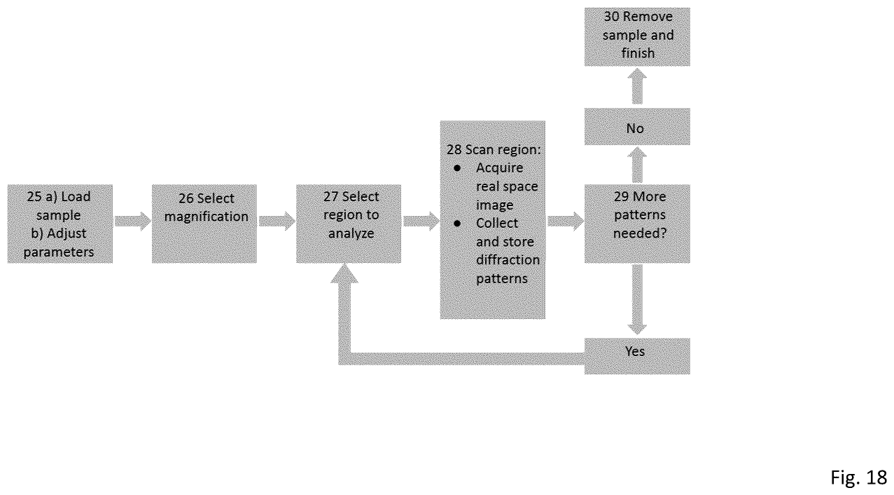

FIG. 17 depicts components of the system in accordance with an illustrative embodiment. The system includes a materials characterization system 4 in which a material sample 5 to be analyzed is placed in a stage. The characterization system can be an electron microscope, particularly a scanning electron microscope, and is capable of collecting both diffraction patterns and real space images from areas of the sample 5. The characterization system may have a spectroscopic detector, like an energy dispersive spectroscopy (EDS) detector, which can be used to characterize the chemical composition of the sample. The characterization system 4 also has access to a memory storage unit 6, so that the control system can acquire and save images from the sample. The characterization system is connected to a control system 1 (that includes one or more processors), which is in turn connected to a user interface 3 that contains a screen, keyboard, etc. allowing input from human users. The user interface contains a display (e.g., liquid crystal display, light-emitting diode display, etc.) of a real space image of the sample, which updates in real time, depicting the portion of the sample that is being scanned by the characterization system 4. The control system 1 also contains a program (or application) that can calculate for any point on the real space image the corresponding position on the sample with respect to a predefined origin, and can control the electron beam and stage to scan over an arbitrary location on the sample 5. In one embodiment, the user interface can also include a printer that is used to print results of an analysis, including the identified characteristics of the samples being analyzed.

The interface 3 of the control system 1 also contains buttons and knobs to adjust different settings on the characterization system 4, such as the sample stage position and magnification. The control system 1 also has a graphical processing unit (GPU) 2 that accelerates any image processing for the sample image. There is also a separate computing system 7 that contains an interface 9, graphical processing units (GPUs) 10, multiple CPUs 8, and other hardware necessary for computations. In an illustrative embodiment, experimental diffraction patterns are acquired through the characterization system 4, and simulated diffraction patterns are generated through the separate computing system 7.

FIG. 18 is a summary of the workflow for acquiring experimental patterns by the system in accordance with an illustrative embodiment. First, the sample is loaded into the characterization system 25. Through the control system interface 3, the user then performs calibration and adjusts experimental settings, such as spot size, accelerating voltage, and beam current (operation 26). Next, in operation 27, the user selects an area of the sample that is to be scanned by the characterization system 4. The area can be selected through an arbitrary method, and diffraction patterns are then collected from the area.

The diffraction patterns can be collected from modalities such as transmission kikuchi diffraction or electron backscattered diffraction, and the real space images can be from modalities like forescattered or backscattered electron images. Accordingly, the characterization system may contain a sample stage, an EBSD or TKD detector, and a forward scattered electron detector. For characterizing samples created by combinatorial synthesis, TKD may be preferred for its higher spatial resolution. After collection, the patterns can be stored in a separate memory unit 6, such as in the hardware of the control system or in a computing cluster. The collected patterns are stored as a 2D image. Their respective positions on the sample may also be saved as metadata.

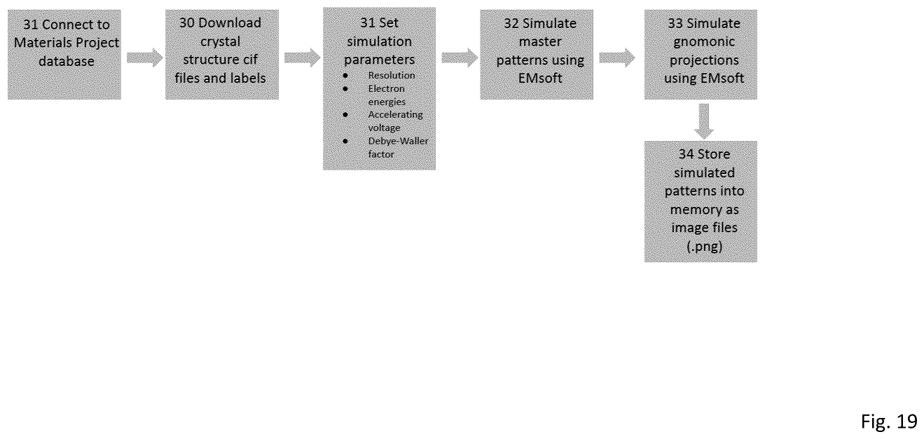

Next, high-throughput diffraction simulations are performed. FIG. 19 depicts a workflow for conducting high-throughput diffraction simulations in accordance with an illustrative embodiment. The computing system 7, which may or may not share the same hardware as the control system 1, contains the hardware for training neural networks as well as running high-throughput computer simulations, and is connected to a user interface 9 that may or may not be the same as that of the control system. First, in operation 29, through the interface, the user obtains a dataset of different inorganic solid crystal structures, either from the internet or pre-downloaded into the storage of the computing system 7. For each crystal structure, the obtained crystal structure data contains information about the unit cell parameters, like the lattice parameters and lattice angles, its space group, and the positions of atoms in the unit cell as well as their atomic numbers. The crystal structure data can be encoded in a variety of formats, such as a CIF file or a .xtal file.

The user, through the interface 9, can add or remove any arbitrary crystal structure from the dataset. For example, the user can remove crystal structures that are above a certain lattice parameter, or that have a certain number of elements in its chemical composition. The user can also remove crystal structures where the difference in atomic number between the element with the highest atomic number and the lowest atomic number is either above and/or below a certain threshold, since these types of crystal structures may be challenging to classify and can be detrimental to model performance. The user can also remove crystal structures with a certain structure prototype that are anticipated to cause misclassifications and which are known to be absent in the experimental sample and/or present in the experimental training data. Additionally, the user can also have the option to upload custom crystal structure data, in case the crystal structure data from online databases is not satisfactory. Next, in operations 30, for each crystal structure, a ‘label’ will also be obtained, which is a value for a certain property of interest that can be inferred from diffraction patterns obtained from a material with the crystal structure. These descriptors can range from symmetry descriptors (e.g. Bravais lattice, space group, point group, strukturbericht prototype, etc.) to structural descriptors (e.g. lattice parameter, chirality) to physical descriptors (e.g. polarity).

These labels can either be obtained from a database, or the user can also use a separate program that can calculate the property of interest using the crystal structure data as input, which can be installed into the computing system 7. These labels can also be arbitrarily modified by the user. For example, when training a neural network to classify crystal symmetry, for some crystal structures, if the signal from the diffraction pattern is anticipated to yield a different crystal symmetry when analyzed from the true crystal symmetry of the structure, then the crystal structure label may be changed to the expected crystal symmetry rather than the true crystal symmetry.

For example, patterns can be relabeled to the crystal symmetry obtained after all the atoms in the crystal structure are changed to the same atomic number. Certain phases with superstructures can also be relabeled to the crystal symmetry of the substructure, corroborating with manual pattern indexing results after bands that are difficult to detect are excluded. Such bands may include superstructure reflections resulting from chemical ordering or other higher ordering that increases that lattice parameter. Weak superstructure reflections can also be excluded based on a an intensity threshold relative to primary band features and/or based on a signal-to-noise or detectability cutoff that is typical for the instrumentation being used. In one embodiment, if there are multiple bands with the same band angle and locations but different widths, the highest intensity band can be used, which will generally be the widest as well, while the narrower ones will likely have resulted from compositional ordering or other higher ordering which is desirable to eliminate from the symmetry determination.

Next, the user performs diffraction simulations for each of the downloaded crystal structures. First, in operation 31, the user specifies different simulation parameters, such as the resolution, accelerating voltage, and sample-detector geometry, which are relevant to the used experimental characterization system. After specifying simulation parameters, the computing system performs simulations of diffraction patterns for the selected dataset of crystal structures. If the diffraction modality used is kikuchi diffraction, the simulations may be performed using existing software like EMsoft or kikuchipy which are capable of performing kikuchi diffraction simulations. For these examples, for an arbitrary input crystal structure, the software first simulates an isotropic representation of the scattered electrons, known as a master pattern, which is shown in operation 32.

In operation 33, gnomonic projections from a section of the master pattern are simulated, which are the final outputs of the simulation and represent an obtained diffraction pattern from the appropriate characterization system. This is done using information about the sample-detector geometry provided by the user. Multiple gnomonic projections may be simulated from the same crystal structure but with different crystal orientations, which result in distinct diffraction signals. These crystal orientations can be sampled using any arbitrary method, such as from a uniform distribution over a sphere, or from the fundamental symmetry zone of the crystal structure. Finally, in operation 34, the simulated patterns are stored into the memory of the computing system 7, along with their respective labels which were given for the crystal structures. For each crystal structure that is simulated, it is important to perform multiple simulations to account for variations in experimental pattern quality. For example, some patterns may have a lower quality from defects or thermal vibrations. Thus, for each crystal structure to be simulated, it is necessary to simulate it twice: once with the assumption of low amounts of thermal vibrations, and once incorporating a higher amount of thermal vibrations, whose exact value may vary depending on the quality of the experimental patterns.

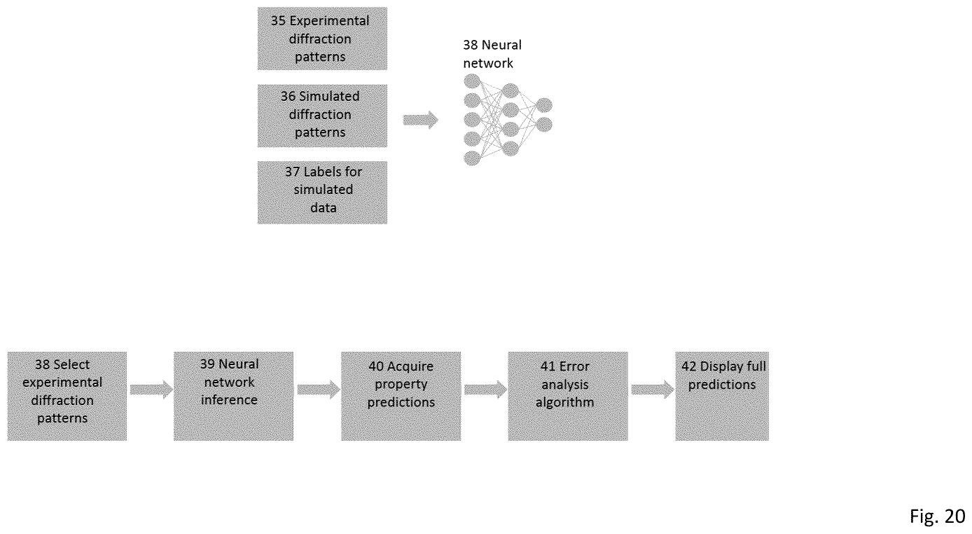

Next, the computing system 7 begins neural network training and classification of the diffraction patterns. FIG. 20 depicts workflows for neural network training (upper) and classification of diffraction patterns (lower) in accordance with an illustrative embodiment. The training process uses a combination of the simulated patterns 36, the labels for the simulated patterns 37, and the collected experimental patterns (or a subset of them) 35 to train a neural network 38 to classify the experimental patterns into the different descriptors for which labels were provided for the simulated patterns. The neural network 38 can be trained through unsupervised domain adaptation techniques, which allows simultaneous training on experimental data for which labels are not provided and simulated data with labels provided.

A number of different algorithms for unsupervised domain adaptation from the literature can be used. In one implementation, the neural network can be trained using a Maximum Classifier Discrepancy method. This method involves a generator backbone that serves as a feature extractor from the input diffraction patterns, and two classifiers that output predictions using the output of the feature extractor as input. Training of the neural network may involve multiple iterations. In each iteration, the neural network updates its weights such that the two neural networks, which are formed by combining the generator with each of the classifiers, minimize cross-entropy classification loss calculated from the neural network predictions on the simulated patterns and their labels. In the next step of the iteration, experimental diffraction patterns are input into the neural network, and their outputs are obtained. The two classifiers then update their weights in order to minimize a discrepancy loss, which is calculated from the classifier outputs for each diffraction pattern. The generator then updates its weights to maximize the same discrepancy loss. Neural network training includes several iterations of the above operations, and the exact number of iterations is set by the user. Any optimization algorithm for updating the weights can be used, and can be pre-selected by the user, such as Adam, stochastic gradient descent, etc. After each iteration, a new file containing the weights of the generator and two classifiers are saved in the memory unit of the computing system 7. The user also has the option to specify before training the exact frequency at which the model weights are stored, such that the weights are only saved every N iterations.

Other neural network training algorithms may also be employed, such as those designed to handle class imbalance or label shift, and there may be an option for the user to either manually choose a particular training algorithm to use, or to upload their own training algorithm.

After training for the specified number of iterations, the resulting neural network(s) 38, including the generator and one of the classifiers, performs inference for either all or a subset of the experimental patterns 35 that were previously acquired by the characterization system 4. The user may also specify an exact training iteration number from which neural network weights are used. To ensure model accuracy, it may be desirable to train multiple neural networks independently on the dataset to create a model ensemble. Then, when making predictions on new experimental patterns, the predictions from each individual model can be aggregated, and the most common prediction could be considered as the final prediction.

The models can also be trained in a quasi-supervised fashion. Experimental patterns could be acquired from phases with known compositions and symmetries, and then used to train an arbitrarily large number of MCD models. Then, models that yield a low accuracy on the experimental training data could be excluded from the model ensemble. In the experimental training data, patterns from different phases can be oversampled or undersampled, in order to ensure the class distribution in the experimental data is sufficiently balanced and achieve high accuracy on the experimental training data.

The computing system 7 can also allow the user to display the neural network prediction for any pattern collected from the sample, in operation 40. Optionally, in operation 41, the computing system 7 may also be programmed to compute possible alternative candidates in case the initial prediction is incorrect, and display these alternatives in operation 42. For example, if the descriptor is the space group, the program may use a logic-based algorithm incorporating crystallographic concepts to determine the subgroups or supergroups of the predicted space groups. Then, in operation 42, the system could display the subgroups as possible next best predictions, if there is any uncertainty with the first prediction. The system may also create a 2-D map that spatially encodes each neural network prediction.

In summary, after the neural network is trained, the system can use the trained neural network to predict the crystal symmetries of patterns in a new EBSD dataset. A 2-D area of a sample can be scanned in a rectangular grid, and a diffraction pattern is collected from each pixel in the grid. Then, the neural network makes a prediction for each diffraction pattern, which can then be used to create a 2-D map of symmetry predictions. In one embodiment, the system maps to spatially resolved orientations and/or phase/symmetry labels over the scanned region. In another embodiment, the system can convert the output into probability/confidence maps, and any of the outputs can be made available for visualization or downstream automated analysis by displaying results on a display, printing results using a printer, providing the results to remote systems, etc.

In some implementations the final trained neural network 38 can be installed into the control system 1 to be used to classify diffraction patterns simultaneously when the characterization system 4 is acquiring patterns from new samples. In some variations, diffraction patterns could be sequentially collected and aggregated from multiple samples before running diffraction simulations and training the classification neural network 38.

When collecting diffraction patterns, many patterns acquired from the same area of the sample may be very similar, such that their analysis would be redundant. Thus, in another variation, each new acquired diffraction pattern may have its similarity calculated with one or more diffraction patterns acquired before it. If the similarity surpasses a predetermined threshold, then the diffraction pattern may be either discarded or flagged as such in its metadata to be possibly excluded during analysis. When collecting diffraction patterns, a pattern quality metric may be calculated for each acquired pattern, and patterns whose pattern quality is determined to be below a certain threshold may be discarded or flagged either for further analysis or so that they are not included in neural network training.

As discussed herein, determining the crystal structure of a material sample can be crucial for determining its functional properties. Current electron backscatter diffraction (EBSD) and transmission Kikuchi diffraction (TKD) methods are often used for crystal structure analysis, where diffraction patterns are collected from a material sample using an electron microscope and then analyzed. After pattern acquisition, methods like Hough indexing or dictionary indexing are used for analyzing the crystal structure. However, as discussed herein, these methods require a small list of different phases/crystal structures that the material sample contains, so that the experimental patterns can be matched to a best-fitting crystal structure. This renders them ineffective for characterization after high-throughput synthesis, since a very large number of distinct and previously unknown crystal structures may be present. The methods and systems described herein circumvent this challenge by using a neural network to directly extract information about the crystal structure from a diffraction pattern without matching it to any particular material phase. This system is appropriate for characterizing samples from high-throughput synthesis, because the millisecond-scale prediction time of neural networks enables the analysis of a high volume of patterns.

Determining the crystal structure of a material sample is crucial for solving R&D problems and discovering new materials. In particular, recent advances in material synthesis are able to synthesize up to millions of different materials at once, on a small enough area so that all the materials can be put under a microscope or any other characterization system at once. To determine which materials are the most useful for any particular application, it is necessary to be able to determine information about the crystal structures of the materials. This is challenging because after high-throughput synthesis, there may be millions of distinct materials that need to be measured, and crystal structure analysis methods thus need to be very fast. To facilitate the analysis, transmission Kikuchi diffraction (TKD) patterns can be collected and analyzed from an SEM, which would be less time-consuming because of the SEM's large field-of-view.

With the proposed system, a neural network classifies the crystal symmetry from transmission Kikuchi diffraction patterns simultaneously while patterns are being collected, yielding crystal structure in formation for a very large number of different material samples in a shorter time frame. With information about the crystal symmetry determined for the materials, downstream calculations of the exact crystal structure and other material properties can be facilitated. This neural network method also enables the classification of crystal structures in a faster manner than other frequently used methods. In particular, conventional methods for determining the crystal structure from a TKD pattern, such as Hough indexing, spherical indexing, and pattern matching, rely on the user to input a list of crystal structures that are already present in the sample. However, for millions of distinct, possibly never before synthesized materials, the crystal structures are unknown and cannot be narrowed down to a small list of structures. As such, these methods are not known to be reliable, and can be time-consuming as individual analysis needs to be repeated for each theoretically possible crystal structure whenever a new collection of diffraction patterns is collected. On the other hand, a neural network, once trained as described herein, can give an almost instantaneous prediction of crystal symmetry for a diffraction pattern, which can enable the crystal structure to be deduced. Other present methods for analyzing the crystal structure from a diffraction pattern that do not require a list of candidate crystal structures involve manual analysis and are enabled by software like CALM or EBSDL, but these methods are also too time-consuming for analyzing a diffraction dataset from a large number of novel materials.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A materials characterization system comprising:
a memory configured to:
store training data that is based on dynamic simulations of crystal structures of materials, wherein the training data includes one or more labels; and
store a plurality of experimental diffraction patterns, wherein the experimental diffraction patterns are unlabeled; and
a processor operatively coupled to the memory and configured to train a convolutional neural network (CNN) based on the stored training data and the experimental diffraction patterns, wherein the CNN is trained to provide a space group classification for an unknown diffraction pattern.

2. The system of claim 1, further comprising an electron backscattered diffraction (EBSD) system that generates the experimental diffraction patterns.

3. The system of claim 1, further comprising a transmission Kikuchi diffraction system that generates the experimental diffraction patterns.

4. The system of claim 1, wherein the training data originates from a materials information database.

5. The system of claim 1, wherein the CNN is trained through unsupervised domain adaptation.

6. The system of claim 5, wherein the unsupervised domain adaption comprises maximum classifier discrepancy.

7. The system of claim 1, wherein the experimental diffraction patterns include a same set of classes that are present in the training data.

8. The system of claim 1, wherein the processor is configured to adjust crystal symmetry labels of the stored training data based at least in part on diffraction patterns within the stored training data.

9. The system of claim 8, wherein the crystal symmetry labels are adjusted based on exclusion of weak superstructure reflections.

10. The system of claim 8, wherein the crystal symmetry labels are adjusted based on visual analysis that is performed by the processor based on a crystal symmetry of a diffraction pattern corresponding to a crystal structure in which all atoms within a unit cell are computationally assigned an identical atomic number.

11. The system of claim 1, wherein the processor artificially resamples the experimental diffraction patterns to keep a class distribution balanced during training with unsupervised domain adaptation.

12. A method for characterizing materials, the method comprising:
storing, in a memory of a computing system, training data that is based on dynamic simulations of crystal structures of materials, wherein the training data includes one or more labels
storing, in the memory, a plurality of experimental diffraction patterns, wherein the experimental diffraction patterns are unlabeled;
training, by a processor operatively coupled to the memory, a convolutional neural network (CNN) based on the stored training data and the experimental diffraction patterns; and
using the trained CNN to provide a space group classification for an unknown diffraction pattern.

13. The method of claim 12, further comprising generating the experimental diffraction patterns with an electron backscattered diffraction (EBSD) system.

14. The method of claim 12, further comprising generating the experimental diffraction patterns with a transmission Kikuchi diffraction system.

15. The method of claim 12, further comprising obtaining the training data from a materials information database.

16. The method of claim 12, wherein the training comprises training the CNN through unsupervised domain adaptation.

17. The method of claim 16, wherein the unsupervised domain adaption comprises maximum classifier discrepancy.

18. The method of claim 12, further comprising adjusting, by the processor, crystal symmetry labels of the stored training data based at least in part on visual analysis of diffraction patterns within the stored training data.

19. The method of claim 18, further comprising performing, by the processor, the visual analysis based on a crystal symmetry of a diffraction pattern corresponding to a crystal structure in which all atoms within a unit cell are computationally assigned an identical atomic number.

20. The method of claim 12, further comprising artificially resampling, by the processor, the experimental diffraction patterns to keep a class distribution balanced during training with unsupervised domain adaptation.
